(19) United States
(12) Patent Application Publication
(10) Pub. No.: US 2007/0098735 A1
(43) Pub. Date: May 3, 2007

(54) Methods for the Elimination of Pathogens and Other Particulate Agents

(76) Inventor: Rajiv Y. Chandawarkar, Farmington, CT (US)

Correspondence Address: CANTOR COLBURN, LLP, 55 GRIFFIN ROAD SOUTH, BLOOMFIELD, CT, 06002, US

(21) Appl. No.: 11/554,283

(22) Filed: Oct. 30, 2006

Related U.S. Application Data
(60) Provisional application No. 60/731,654, filed on Oct. 29, 2005.

(51) Int. Cl. A61K 39/12; A61K 39/02; A61K 39/00

(52) U.S. Cl. 424/185.1; 424/204.1; 424/234.1

(57) ABSTRACT

Disclosed herein are methods of treating infections, wounds, and exposure to particulate agents, comprising administering a heat shock protein (HSP) such as HSP70. The HSP may be uncomplexed to an antigenic peptide. Uncomplexed HSPs have been shown to activate phagocytosis by macrophages, and are thus particularly useful for the rapid clearance of harmful particulate agents including pathogenic organisms and particulate agents such as particulate pollutants.

### CROSS-REFERENCE TO RELATED APPLICATIONS

This application claims the benefit of U.S. Patent Application Ser. No. 60/731,654, filed Oct. 29, 2005, which is incorporated by reference herein in its entirety.

### BACKGROUND

Heat Shock Proteins (HSPs) are abundant intracellular molecules that are readily released by cell lysis following injury or infection, where they exhibit broad immunoactive properties. HSPs have been shown to play a major role in macrophage activation, preparing the host for defense. Immunologically, HSPs bind several macrophage-surface receptors and upregulate key antigen-specific and non-specific functions, including tumor rejection, cytokine release, and upregulation of co-stimulatory molecules. Within the confines of the cell, HSPs are chaperones and facilitate protein synthesis and breakdown. They are expressed in all cells in all forms of life and in a variety of intracellular locations: in the cytosol (HSP70 and HSP90), nuclei, endoplasmic reticulum (gp96), and mitochondria, for example. In addition to their ubiquity, the HSPs constitute the single most abundant group of proteins inside cells. They are expressed in cells under normal un-stressed conditions, and their expression can be powerfully induced to much higher levels as a result of heat shock or other forms of stress, including exposure to toxins, oxidative stress, glucose deprivation, and the like, leading up to cell lysis which releases large quantities of HSPs into the extracellular milieu. Approximately ten families of HSPs are known, and each family comprises anywhere from one to five or more closely related proteins. Since their discovery, an increasing array of functions such as folding and unfolding of proteins, degradation of proteins, assembly of multi-subunit complexes, thermotolerance, buffering of expression of mutations, and others have been attributed to HSPs.

When isolated from, for example, a eukaryotic tissue, HSPs comprise bound peptide molecules. HSP-peptide complexes have been employed to treat intracellular pathogens as in U.S. Pat. No. 6,048,530. Vaccine compositions were prepared by isolating HSP-peptide complexes from eukaryotic cells infected with the pathogen thus producing a complex wherein the complex comprises a peptide from the pathogen. The HSP-peptide complexes stimulate an immune response in a mammal infected with the same pathogen. It is the HSP-peptide complex that was shown to induce a cytotoxic T cell response against the pathogen.

Also, as described in U.S. Pat. No. 6,139,841, HSP70 bound to an antigenic peptide can be used to treat an infectious disease cased by, for example, a virus, bacterium, protozoan, fungus, or parasite. HSPs have also been described as useful to treat cancer and infectious diseases by administering a polynucleotide encoding an HSP as described in U.S. Patent Publication No. 2002/0198166. In the examples, treatment of tumor cells with polynucleotides expressing HSPs was shown. In addition, as described, for example, in U.S. Pat. No. 6,475,490, HSPs bound to peptide molecules may be employed to stimulate tissue repair in a mammal. The types of damaged tissues to be repaired include lesions, traumatic damage (e.g., surgery, injury) and disease damage. In the examples, gp96-peptide complexes were shown to accelerate wound healing in dorsal skin wounds in a mouse model.

There remains a need in the art for the identification of additional cellular roles and therapies involving heat shock proteins.

### SUMMARY

In one embodiment, a method of promoting pathogen elimination from a vertebrate organism comprises administering a phagocytosis-stimulating amount of an uncomplexed heat shock protein to the vertebrate organism in need of such administration, wherein administering is therapeutic or prophylactic.

In another embodiment, a method of reducing infection, necrotic tissue, or both at a wound site in a vertebrate organism comprises administering a phagocytosis-stimulating amount of an uncomplexed heat shock protein to the vertebrate organism in need of such administration, wherein the vertebrate organism has an infected wound, a chronic wound comprising necrotic tissue, or a combination thereof.

In yet another embodiment, a method of promoting particulate agent elimination from a vertebrate organism comprises administering a phagocytosis-stimulating amount of an uncomplexed heat shock protein to the vertebrate organism after exposure or suspected exposure to the particulate agent.

### BRIEF DESCRIPTION OF THE FIGURES

FIG. 1: Macrophage-mediated phagocytosis is modulated by HSPs. The ability of macrophage cell lines (as indicated) to phagocytose either Alexa Fluor® 488-labeled inert microspheres, Saccharomyces cerevisiae (S. cerevisiae) or Escherichia coli (E. coli) in absence or presence of each the HSPs indicated (HSP70, HSP90 or gp96) or mouse serum albumin (MSA) was tested in vitro.

FIG. 2: HSP70-mediated phagocytosis is specific, titratable, actin dependent and independent of protein synthesis and HSP peptides. (A) Macrophages were treated with HSP70 or non-HSP controls (as indicated) to examine their specific effects on phagocytosis of Alexa Fluor® 488-labeled S. cerevisiae. (B) Macrophages were treated with HSP70 (doses indicated) and subsequently tested for their ability to phagocytose Alexa Fluor® 488-labeled S. cerevisiae. (C) Representative microphotographs (10×) of macrophages (treated for minutes as indicated). (D) Macrophages were pre-treated with HSP70, washed free of residual HSP70, administered yeast and tested in a phagocytosis assay. (E) HSP70-coated S. cerevisiae was prepared by co-incubating Alexa Fluor® 488-labeled S. cerevisiae with HSP70 and washed until free of unbound HSP70. (F) Macrophages were treated with HSP70 under (i) conditions that prevent actin-mediated cytoskeletal changes or (ii) in the presence or absence of cyclohexamide and subjected to a phagocytosis assay or (iii) Phagocytosis-enhancing effects of adenosine triphosphate (ATP)-treated HSP70 (peptide free) was compared with that of adenosine diphosphate (ADP)-purified HSP70 (with peptides).

FIG. 3: HSP70-mediated phagocytosis is independent of lipopolysaccharide (LPS). (A) Macrophages were treated with LPS in serum-free conditions and resultant tumor necrosis factor-α (TNF-α) production was measured. (B) Macrophages maintained in serum-free conditions were treated with either HSP70 or of LPS (as indicated), or heat-denatured HSP70 and a phagocytosis assay was performed.

FIG. 4: HSP70-macrophage interaction occurs on the lipid raft (LR) microdomain of macrophage plasma membranes. (A) Macrophages treated with exogenous horseradish peroxidase (HRP)-labeled HSP70 were washed, and lysed. (B) Similar to the conditions in (A) macrophages were treated with HSP70-HRP and the LR-fractions were purified. These fractions were tested for the presence of: HSP70; GM1 ganglioside (GM1); and assayed for the amount of cholesterol. (C) The influence of the LR-integrity on HSP70-mediated phagocytosis was tested by treating macrophages with nystatin or methyl-β-cyclodextrin (MCD) (both drugs disrupt LRs) and a phagocytosis assay was performed. (D) RAW264.7 macrophages were treated with HSP70 in the presence of varying doses of MCD (as indicated).

FIG. 5: Increased HSP70-mediated phagocytosis enhances antigen presentation. (A) Ovalbumin (OVA)-coated yeast were administered to macrophages treated with HSP70 or control proteins in the presence or absence of cytochalasin-D and the resultant CD4+ proliferation was measured using fluorescence activated cell sorting (FACS) as an indicator of the amount of the OVA peptide presented in context of major histocompatibility complex (MHC)-II antigen presentation. (B) Concurrent production of interferon gamma (IFNγ) by the CD4+ T cells was quantified using enzyme-linked immunosorbent assay (ELISA) as a measure of their effector function.

FIG. 6. Exogenous administration of HSPs accelerates wound healing. (A) Schematic representation of the wound healing study design. (B-D) Comparison of the percent wound closure of mice wounded on day 0 and treated as indicated.

FIG. 7. The effect of HSPs on wounds is protein-specific and dose titratable. Mice were wounded and treated with either phosphate buffered saline (PBS), LPS, Uric Acid, Phosphorylase B, MSA, HSP70, HSP90, or gp96.

FIG. 8. The pro-inflammatory cytokine profile of HSP-treated wounds. (A-C) Levels of proinflammatory cytokines in wound tissue. Mice were treated with PBS, LPS, MSA, HSP70, HSP90 or gp96.

FIG. 9. HSP70 exerts its effect on wound healing via macrophages in a specific and titratable fashion. (A) Comparison of the percent wound healing of mice wounded on day 0 and injected with 400,000 macrophages pre-treated with either PBS (n=5), LPS (n=5), MSA (n=5), or HSP70 (n=5) on days 0, 2, 4 post surgery. Data is plotted as average percent wound closure ±SEM on days indicated. (B) Mice wounded on day 0 were adoptively transferred with 4,000 (n=5), 40,000 (n=5) or 400,000 (n=5) macrophages each group treated with HSP70, or PBS (n=5) on days 0, 2, and 4 post surgery

FIG. 10. HSP70 accelerates wound healing by upregulating macrophage-mediated phagocytosis. (A) Macrophages were treated with HSP70, LPS or MSA (n=4 mice each group) and administered wound debris. (B) Mice were treated with Gadolinium Chloride (GdCl₃) on day −1 and day 0 pre-surgery. Mice were subsequently wounded and treated with either HSP70 or PBS on day 0 and percent wound closure was measured.

FIG. 11: HSP70 induces phagocytosis via its interaction with Toll-like receptor-7 (TLR7). (A) RAW264.7 macrophages were treated with either one of the known ligands of TLR7 given alone as shown in (A) or administered (B) in the presence of TLR7-ligands as indicated and assayed in a phagocytosis assay. (C) RAW264.7 cells were treated with HSP70 in the presence of varying doses of Loxoribine (LRB), a TLR7-ligand and a phagocytosis assay was performed. (D) RAW264.7 cells were treated with either HSP70 alone or with HSP70 that had been pre-treated with RNAase or heat denatured (as indicated) and a phagocytosis assay was performed.

### DETAILED DESCRIPTION

The present invention includes the use of heat shock proteins (HSPs) for the elimination of pathogens or other harmful particulate agents such as particulate pollutants from a vertebrate organism exposed to such an agent by stimulating phagocytosis by macrophages. Also included is the use of HSPs to stimulate macrophages to phagocytose necrotic debris in wounds. The HSPs can be administered therapeutically to a vertebrate organism with an infection by a pathogen, to a vertebrate organism that has been exposed to a pathogen or particulate agent, to a vertebrate organism suspected of exposure to a pathogen or particulate agent, or prophylactically to a vertebrate organism at risk of future exposure to a pathogen or particulate agent. The HSPs can also be administered to a vertebrate organism having a wound site such as an infected and/or necrotic wound site.

Previously, HSP-peptide complexes had been employed in methods such as therapy for infections. It has been unexpectedly discovered that uncomplexed HSPs activate phagocytosis by macrophages, and are thus particularly useful for the rapid clearance of harmful particulate agents including pathogenic organisms, other particulate agents such as particulate pollutants, and necrotic debris. A major advantage of this discovery is that the therapy is non-specific, that is, a peptide specific for the pathogen to be treated is not required. Thus, the pathogen need not be identified prior to treatment with uncomplexed HSPs. Treatment with uncomplexed HSPs can be used as a first line treatment that can be administered even prior to identification of the particular pathogen or particulate pollutant. A first line treatment is the first type of therapy given for a condition or disease. Use of uncomplexed HSPs to stimulate an immune response has particular utility in the treatment of NIAID category A, B, and C priority pathogens; unidentified pathogens; emergent pathogens; antibiotic-resistant pathogens; pathogens that are difficult to detect; or a combination comprising one or more of the foregoing pathogens.

The uncomplexed HSPs are also particularly useful for the treatment of systemic infections such as infections that are disseminated through the circulatory system. Systemic infections, also called disseminated infections, can be caused by bacteria and bacteria-like prokaryotes, fungi, protozoa, and viruses. The uncomplexed HSPs are also useful in the treatment of deep tissue infections such as those in infected body cavities and deep-rooted tissue infections. The uncomplexed HSPs are also particularly useful in the treatment of invasive fungal infections.

The present invention also relates to the use of HSPs to reduce infection and/or necrotic tissue at a wound site in a vertebrate organism. An advantage of the use of uncomplexed HSPs to treat a wound is that the infectious agent need not be identified prior to treatment. The stimulation of phagocytosis by macrophages can result in accelerated clearance of infectious agents as well as cellular debris and foreign matter, ultimately leading to accelerated wound healing. Wound sites include tissues damaged by traumatic injury such as an automobile accident, tissues damaged by severe diabetic infection, and tissues damaged by candidal infections. In one embodiment, the wound site is a chronic wound site comprising necrotic tissue.

In yet another embodiment, HSPs are used to treat vertebrate organisms that have been exposed to other particulate agents such as particulate pollutants. Vertebrate organisms can be exposed to particulate pollutants by, for example, inhalation, ingestion, or through the skin such as through an injury. Particulate environmental pollutants include, for example, asbestos, cadmium, mercury, silica, coal dust, carbon, tin oxide, beryllium, stone dust, graphite, and combinations comprising one or more of the foregoing pollutants. Particulate agents also include biological weapons, which typically include a biological agent and optionally a carrier suitable, for example, for aerosol administration. The uncomplexed HSPs are thus also useful in bioterrorism and biological warfare applications. An advantage of the use of uncomplexed HSPs is that the phagocytosis process stimulated by the HSPs is nonspecific and can be used to treat exposure even when the particulate agent is unknown.

The invention is based at least in part on the novel identification of uncomplexed heat shock proteins, such as HSP70, as a mammalian agent that can activate phagocytosis, a primitive macrophage function. Soon after uncomplexed HSP70 treatment, macrophages are stimulated to internalize a variety of particulate materials including gram positive and negative bacteria (e.g., Staphylococcus aureus, E. coli), fungi (e.g., Saccharomyces cerevisiae, Candida albicans) and inert particles (e.g., polystyrene microspheres).

It has been shown by the inventors herein that HSP70 interacts with lipid raft (LR) microdomains on the macrophage-cell surface. This binding may occur on a specific LR-bound receptor, structural components of the LR, or HSP70 may be taken up by a different macrophage receptor and binds the inner leaflet of the LR. Although the physical existence of lipid rafts has been questioned by some, these results define the influence of LRs on HSP-stimulated phagocytosis. Disruption of the LRs partially abrogates the HSP-mediated effects on phagocytosis. Without being held to theory, it is believed that lipid rafts could provide a platform for the interaction between the HSP70 and the phagocytic receptors. It is further believed that the nature of further signaling is dependent upon endocytosis (data not shown). It is possible that the entire HSP70-LR complex is endocytosed and that this complex actually activates the phagocytic receptors. The fact that cyclohexamide is unable to block HSP-mediated phagocytosis indicates that new protein synthesis is not required, partly explaining the quick onset of HSP-mediated phagocytosis. Disruption of the LRs abrogates the HSP-mediated phagocytosis, suggesting that LRs provide a platform for HSP70 and subsequent activation of the phagocytic receptors. The quick onset of phagocytosis, within minutes of HSP70-treatment, and the non-dependence on synthesis of new proteins (cyclohexamide-independent) suggests that HSP70-mediated enhancement of phagocytosis occurs via a short signaling pathway, possibly membrane-bound, and does not involve a gene upregulation.

The inventors herein have discovered that HSP70, a phylogenetically conserved molecule, plays an important role in the innate host response to pathogens. Through the extracellular release of 100 μg/ml HSP70, even a relatively small amount of tissue lysis, either from infection or injury could potentially elicit macrophage phagocytosis. It is important to note that the HSP70 is able to stimulate phagocytosis whether it carries peptides on it or not. Both ADP- and ATP-purified HSP70 are equally capable of stimulating phagocytosis. Since the non-specific responses to HSPs are independent of peptides chaperoned by HSPs, they act similarly to bacterial lipopolysaccharides (LPS). Similar results are expected with the other HSPs.

The significance of the discovery that extracellular HSPs are recognized by LR-microdomain of the macrophage is that this recognition triggers phagocytosis, a primal mechanism of self-defense. Modulating the HSP70-LR interaction presents an opportunity to intervene at the level of host-pathogen interface providing a therapeutic tool for emerging infections, especially where conventional treatment with antibiotics is ineffective (antibiotic resistance) or unavailable (rapidly spreading, endemic). Sequentially, HSP70-mediated phagocytosis leads to increased antigenic processing and presentation to CD4+ T lymphocytes, which proliferate and release interferon-γ (IFN-γ). In this context, HSP70-LR interaction plays an important role, not only in clearing invading agents, but in processing and presenting their antigens to the host immune system. The wide range of microbial and non-microbial agents phagocytosed in response to HSP70-LR interaction as well as its remarkably short time of onset presents the opportunity to develop a rapidly deployable therapeutic intervention. Potentially, it could not only facilitate early elimination of the invading agent, but also activate the immune system into a state of heightened preparedness.

It has also been shown herein, in murine models of wound healing, that exogenously added uncomplexed HSPs are powerful agents that influence tissue repair, act rapidly and exert a sustained effect throughout the entire process of healing. This work differs from previous studies in a number of ways. It provides evidence that extracellular presence of exogenously administered HSPs play a role in the process of wound repair through their influence on wound biogenesis. This extends the observations made by others that the intracellular increase in HSP-expression correlated with wound healing. The HSPs influence wound repair when released into the extracellular compartment of the wound where they come into direct contact with macrophages and other cellular components that are essential to the repair process.

The results of this study highlight some of the characteristics of uncomplexed HSP-mediated tissue repair. The rapid onset of HSP-effects on wound healing indicates that HSPs ‘jump-start’ the tissue repair process. Wounds of HSP-treated animals show a dramatic degree of healing within the first few days of treatment and this effect seems to plateau towards the mid and latter portion of the healing process. This result can be explained by the fact that HSPs exert their effects on wounds via early cellular recruits such as macrophages. Phagocytosis is an early event in wound healing. Phagocytic elimination of debris, devitalized tissues, spent neutrophils and pathogen clears the ground for new vital tissues. HSPs accelerate the entire wound healing process by upregulating its early scavenger phase. The activation of macrophages exerts a “domino effect” leading to release of lipid mediators, cytokines, and chemokines that mediate recruitment and activation of additional inflammatory cells.

Phagocytosis is a primal protective cellular function that characterizes the innate immune response to microbial invasion. It is a complex phenomenon implicating several components of the plasma membrane including pattern recognition receptors (PRRs), cytoskeletal elements and lipid rafts. Antigen presenting cells (APCs) act as sentinels of the host that initiate and execute the phagocytic response. These cells are activated in response to certain well defined substances that provide stimulatory signals that characterize injury or infection. APCs, including macrophages, respond either to the presence of pathogens via PRRs or to metabolic, physical or chemical stress, trauma, or other agents that mediate necrotic cell lysis and death.

The invention includes the use of a heat shock protein to stimulate phagocytosis of a particulate agent. While stimulation of phagocytosis has been exemplified with HSP70, it is expected that the methods disclosed herein can be performed with any suitable HSP. Exemplary HSPs include HSP70, HSP90, gp96, HSP47, HSP60, HSP65, HSP 110, HSP 10, HSP 104, HSP40, HSP27, and HSP20. HSPs, which are also referred to interchangeably as stress proteins, are cellular proteins wherein the intracellular concentration increase when a cell is exposed to a stressful stimuli (e.g., elevated temperatures), are capable of binding other proteins or peptides, are capable of releasing the bound proteins or peptides in the presence of adenosine triphosphate (ATP) or low pH, or at least 35% homologous with a cellular protein having the above properties. Suitable HSPs include gp96, HSP90, and HSP70, either alone or in combination with each other. Specifically, the HSPs are human HSPs, although the HSPs may be mammalian, murine, bovine, feline, bacterial, and the like.

The first stress proteins to be identified were the heat shock proteins. As their name implies, HSPs are synthesized by a cell in response to heat shock. To date, three major families of HSP have been identified based on molecular weight. The families have been called HSP60, HSP70 and HSP90, wherein the numbers reflect the approximate molecular weight of the stress proteins in kilodaltons. Mammalian HSP90 and gp96 each are members of the HSP90 family. Many members of these families were subsequently found to be induced in response to other stressful stimuli including, but not limited to, nutrient deprivation, metabolic disruption, oxygen radicals, and infection with intracellular pathogens. It is contemplated that HSPs/stress proteins belonging to all of these three families can be used in the methods described herein.

The major HSPs can accumulate to very high levels in stressed cells, but they occur at low to moderate levels in cells that have not been stressed. For example, the highly inducible mammalian HSP70 is hardly detectable at normal temperatures but becomes one of the most actively synthesized proteins in the cell upon heat shock. In contrast, the HSP90 and HSP60 proteins are abundant at normal temperatures in most, but not all, mammalian cells and are further induced by heat.

Heat shock proteins are among the most highly conserved proteins in existence. For example, DnaK, the HSP70 from E. coli has about 50% amino acid sequence identity with HSP70 proteins from excoriates. The HSP60 and HSP90 families also show similarly high levels of intra family conservation. In addition, it has been discovered that the HSP60, HSP70 and HSP90 families are composed of proteins that are related to the stress proteins in sequence, for example, having greater than 35% amino acid identity, but whose expression levels are not altered by stress. Therefore it is contemplated that the definition of stress protein, as used herein, embraces other proteins, muteins, analogs, and variants thereof having at least 35% to 55%, preferably 55% to 75%, and most preferably 75% to 85% amino acid identity with members of the three families whose expression levels in a cell are enhanced in response to a stressful stimulus.

Heat shock proteins can be uncomplexed or complexed with a peptide such as a peptide antigen. In one embodiment, the heat shock protein is in uncomplexed form. As used herein, uncomplexed HSP refers to an HSP that is not complexed to an antigenic peptide. An advantage of the methods disclosed herein is that phagocytosis stimulated by HSPs is nonspecific and does not require a peptide antigen bound to the HSPs.

In one embodiment, uncomplexed endogenous HPSs are isolated from eukaryotic cells, including but not limited to, tissues, isolated cells, and immortalized eukaryotic cell lines. The tissue source need not be the same as the tissue which is targeted by the subject repair response, for example. Suitable source tissues include, but are not limited to purified lymphocytes, liver, spleen, or another organ of mammalian or non-mammalian origin. Source tissue may be autologous (i.e., from the same individual) or non-autologous. Non-autologous source tissue may be obtained from a cadaver. In an embodiment, uncomplexed endogenous HSPs and endogenous HSPs complexed with antigenic molecules are isolated from tumor cells. In another embodiment, the HSP or HSP-peptide complex is isolated from tissue excised from a human.

In one embodiment, a gp96-peptide complex is purified as follows:

A pellet of eukaryotic cells (e.g., from liver, spleen, or any other suitable organ) is resuspended in 3 volumes of lysis buffer (30 mM sodium bicarbonate buffer (pH 7.5)) and 1 mM phenyl methyl sulfonyl fluoride (PMSF) and the cells allowed to swell on ice for 20 minutes. The cell pellet then is homogenized in a Dounce homogenizer (the appropriate clearance of the homogenizer will vary according to each cell type) on ice until >95% cells are lysed.

The lysate is centrifuged at 1,000×g for 10 minutes to remove unbroken cells, nuclei and other debris. The supernatant from this centrifugation step then is recentrifuged at 100,000×g for 90 minutes. The gp96-peptide complex can be purified either from the 100,000×g pellet or from the supernatant.

When purified from the supernatant, the supernatant is diluted with equal volume of 2× lysis buffer and then mixed for 2-3 hours at 40° C. with Con A-Sepharose®(Pharmacia, Inc., Sweden) equilibrated with PBS containing 2 mM Ca²⁺ and 2 mM Mg²⁺. Then, the slurry is packed into a column and washed with 1× lysis buffer until the OD₂₈₀ drops to baseline. The column is washed with ⅓ column bed volume of 10% α-methyl mannoside (α-MM) dissolved in phosphate buffered saline (PBS) containing 2 mM Ca²⁺ and 2 mM Mg²⁺, the bottom of the column sealed with a piece of parafilm, and incubated at 37° C. for 15 minutes. Then the column is cooled to room temperature and the parafilm removed from the bottom of the column. Five column volumes of the α-MM buffer are applied to the column and the eluate is analyzed by sodium dodecyl sulfate-polyacrylamide gel electrophoresis (SDS-PAGE). Typically, the resulting material is about 60-95% pure, however this depends upon the cell type and the tissue-to-lysis buffer ratio employed. Then the sample is applied to a Mono Q® FPLC ion-exchange chromatographic column (Pharmacia, Inc., Piscataway, N.J.) equilibrated with a buffer containing 5 mM sodium phosphate, pH 7. The proteins are then eluted from the column with a 0-1M NaCl gradient. The gp96 fraction elutes between 400 mM and 550 mM NaCl.

The procedure, however, can be modified by two additional steps, used either alone or in combination, to consistently produce apparently homogeneous gp96-peptide complexes. One optional step involves an ammonium sulfate precipitation prior to the Con A purification step and the other optional step involves DEAE-Sepharose® (Pharmacia, Inc., Piscataway, N.J.) purification after the Con A purification step but before the Mono Q® FPLC step.

In the first optional step, the supernatant resulting from the 100,000×g centrifugation step is brought to a final concentration of 50% ammonium sulfate by the addition of ammonium sulfate. The ammonium sulfate is added slowly while gently stirring the solution in a beaker placed in a tray of ice water. The solution is stirred from about 0.5 to 12 hours at 4° C. and the resulting solution centrifuged at 6,000 rpm (Sorvall SS34 rotor). The supernatant resulting from this step is removed, brought to 70% ammonium sulfate saturation by the addition of ammonium sulfate solution, and centrifuged at 6,000 rpm (Sorvall SS34 rotor). The resulting pellet from this step is harvested and suspended in PBS containing 70% ammonium sulfate in order to rinse the pellet. This mixture is centrifuged at 6,000 rpm (Sorvall SS34 rotor) and the pellet dissolved in PBS containing 2 mM Ca²⁺ and Mg²⁺. Undissolved material is removed by a brief centrifugation at 15,000 rpm (Sorvall SS34 rotor). Then, the solution is mixed with Con A Sepharose® and the procedure followed as before.

In the second optional step, the gp96 containing fractions eluted from the Con A column are pooled and the buffer exchanged for 5 mM sodium phosphate buffer, pH 7, 300 mM NaCl by dialysis, or preferably by buffer exchange on a Sephadex® G25 column (Pharmacia, Inc., Sweden). After buffer exchange, the solution is mixed with DEAE-Sepharose® previously equilibrated with 5 mM sodium phosphate buffer, pH 7, 300 mM NaCl. The protein solution and the beads are mixed gently for 1 hour and poured into a column. Then, the column is washed with 5 mM sodium phosphate buffer, pH 7, 300 mM NaCl, until the absorbance at 280 nM drops to baseline. Then, the bound protein is eluted from the column with five volumes of 5 mM sodium phosphate buffer, pH 7, 700 mM NaCl. Protein containing fractions are pooled and diluted with 5 mM sodium phosphate buffer, pH 7 in order to lower the salt concentration to 175 mM. The resulting material then is applied to the Mono Q® FPLC column equilibrated with 5 mM sodium phosphate buffer, pH 7 and the protein that binds to the Mono Q® FPLC column is eluted as described before.

It is appreciated, however, that one skilled in the art can assess, by routine experimentation, the benefit of incorporating the second optional step into the purification protocol. In addition, it is appreciated also that the benefit of adding each of the optional steps will depend upon the source of the starting material.

When the gp96 fraction is isolated from the 100,000×g pellet, the pellet is suspended in 5 volumes of PBS containing either 1% sodium deoxycholate or 1% octyl glucopyranoside (but without the Mg²⁺ and Ca²⁺) and incubated on ice for 1 hour. The suspension is centrifuged at 20,000×g for 30 minutes and the resulting supernatant dialyzed against several changes of PBS (also without the Mg²⁺ and Ca²⁺) to remove the detergent. The dialysate is centrifuged at 100,000×g for 90 minutes, the supernatant harvested, and calcium and magnesium are added to the supernatant to give final concentrations of 2 mM, respectively. Then the sample is purified by either the unmodified or the modified method for isolating gp96-peptide complex from the 100,000×g supernatant, see above.

The gp96-peptide complexes can be purified to apparent homogeneity using this procedure. About 10-20 μg of gp96-peptide complex can be isolated from 1 g cells/tissue.

In one embodiment, an HSP70-peptide complex is purified as follows:

Initially, cells (e.g., from liver, spleen, or any other suitable organ) are suspended in 3 volumes of 1× lysis buffer consisting of 5 mM sodium phosphate buffer, pH 7, 150 mM NaCl, 2 mM CaCl₂, 2 mM MgCl₂ and 1 mM PMSF. Then, the pellet is sonicated, on ice, until >99% cells are lysed as determined by microscopic examination. As an alternative to sonication, the cells can be lysed by mechanical shearing and in this approach the cells typically are resuspended in 30 mM sodium bicarbonate pH 7.5, 1 mM PMSF, incubated on ice for 20 minutes and then homogenized in a dounce homogenizer until >95% cells are lysed.

Then the lysate is centrifuged at 1,000×g for 10 minutes to remove unbroken cells, nuclei and other cellular debris. The resulting supernatant is recentrifuged at 100,000×g for 90 minutes, the supernatant harvested and then mixed with Con A Sepharose® equilibrated with PBS containing 2 mM Ca²⁺ and 2 mM Mg²⁺. When the cells are lysed by mechanical shearing, the supernatant is diluted with an equal volume of 2× lysis buffer prior to mixing with Con A Sepharose®. The supernatant is then allowed to bind to the Con A Sepharose® for 2-3 hours at 4° C. The material that fails to bind is harvested and dialyzed for 36 hours (three times, 100 volumes each time) against 10 mM Tris-Acetate pH 7.5, 0.1 mM ethylenediaminetetraacetic acid (EDTA), 10 mM NaCl, 1 mM PMSF. Then the dialyzate is centrifuged at 17,000 rpm (Sorvall SS34 rotor) for 20 minutes. Then the resulting supernatant is harvested and applied to a Mono Q® FPLC column equilibrated in 20 mM Tris-Acetate pH 7.5, 20 mM NaCl, 0.1 mM EDTA and 15 mM 2-mercaptoethanol. The column is then developed with a 20 mM to 500 mM NaCl gradient and eluted fractions fractionated by SDS-PAGE and characterized by immunoblotting using an appropriate anti-HSP70 antibody (such as from clone N27F3-4, from StressGen, Victoria, British Columbia, Canada).

Fractions strongly immunoreactive with the anti-HSP70 antibody are pooled and the HSP70-peptide complexes precipitated with ammonium sulfate; specifically with a 50%-70% ammonium sulfate cut. The resulting precipitate is then harvested by centrifugation at 17,000 rpm (SS34 Sorvall rotor) and washed with 70% ammonium sulfate. The washed precipitate is then solubilized and any residual ammonium sulfate removed by gel filtration on a Sephadex® G25 column. If necessary the HSP70 preparation thus obtained can be repurified through the Mono Q® FPLC column as described above for gp96 purification.

The HSP70-peptide complex can be purified to apparent homogeneity using this method. Typically 1 mg of HSP70-peptide complex can be purified from 1 g of cells/tissue.

In another embodiment, HSP70-peptide complexes are purified by an alternate rapid purification process. This improved method comprises contacting cellular proteins with adenosine diphosphate (ADP) or a nonhydrolyzable analog of adenosine triphosphate (ATP) affixed to a solid substrate, such that HSP70 in the lysate can bind to the ADP or nonhydrolyzable ATP analog, and eluting the bound HSP70. A suitable method uses column chromatography with ADP affixed to a solid substratum (e.g., ADP-agarose). The resulting HSP70 preparations are higher in purity and devoid of contaminating peptides. The HSP70 yields are also increased significantly by about more than 10 fold. Alternatively, chromatography with nonhydrolyzable analogs of ATP, instead of ADP, can be used for purification of HSP70-peptide complexes.

By way of example but not limitation, purification of HSP70-peptide complexes by ADP-agarose chromatography is carried out as follows: 500 million cells (e.g., from liver, spleen, or any other suitable organ) are homogenized in hypotonic buffer and the lysate is centrifuged at 100,000×g for 90 minutes at 4° C. The supernatant is applied to an ADP-agarose column. The column is washed in buffer and is eluted with 5 column volumes of 3 mM ADP. The HSP70-peptide complexes elute in fractions 2 through 10 of the total 15 fractions which elute. The eluted fractions are analyzed by SDS-PAGE. The HSP70-peptide complexes can be purified to apparent homogeneity using this procedure.

In one embodiment, an HSP90-peptide complex is purified as follows:

Initially, cells (e.g., from liver, spleen, or any other convenient organ) are suspended in 3 volumes of 1× lysis buffer consisting of 5 mM sodium phosphate buffer (pH7), 150 mM NaCl, 2 mM CaCl₂, 2 mM MgCl₂ and 1 mM PMSF. Then, the pellet is sonicated, on ice, until >99% cells are lysed as determined by microscopic examination. As an alternative to sonication, the cells can be lysed by mechanical shearing and in this approach the cells typically are resuspended in 30 mM sodium bicarbonate pH 7.5, 1 mM PMSF, incubated on ice for 20 minutes and then homogenized in a dounce homogenizer until >95% cells are lysed.

Then the lysate is centrifuged at 1,000×g for 10 minutes to remove unbroken cells, nuclei and other cellular debris. The resulting supernatant is recentrifuged at 100,000×g for 90 minutes, the supernatant harvested and then mixed with Con A Sepharose® equilibrated with PBS containing 2 mM Ca²⁺ and 2 mM Mg²⁺. When the cells are lysed by mechanical shearing the supernatant is diluted with an equal volume of 2× lysis buffer prior to mixing with Con A Sepharose®. The supernatant is then allowed to bind to the Con A Sepharose® for 2-3 hours at 4° C. The material that fails to bind is harvested and dialyzed for 36 hours (three times, 100 volumes each time) against 20 mM sodium phosphate, pH 7.4, 1 mM EDTA, 250 mM NaCl, 1 mM PMSF. Then the dialyzate is centrifuged at 17,000 rpm (Sorvall SS34 rotor) for 20 minutes. Then the resulting supernatant is harvested and applied to a Mono Q® FPLC column equilibrated with a buffer containing 20 mM sodium phosphate, pH 7.4, 1 mM EDTA, 250 mM NaCl, 1 mM PMSF. The proteins are then eluted with a salt gradient of 200 mM to 600 mM NaCl.

The eluted fractions are fractionated by SDS-PAGE and fractions containing the HSP90-peptide complexes identified by immunoblotting using an anti-HSP90 antibody such as 3G3 (Affinity Bioreagents). Hsp90-peptide complexes can be purified to apparent homogeneity using this procedure. Typically, 150-200 μg of HSP90-peptide complex can be purified from 1 g of cells/tissue.

In one embodiment, the HSP is in uncomplexed form. Methods which can be used to separate the HSP and antigenic molecule components of the HSP-antigenic molecule complexes from each other, include, but are not limited to, treatment of the complexes with low pH. The low pH treatment methods can be used, for example, for HSP70, HSP90, or gp96.

By way of example but not limitation, to elute the noncovalently bound antigenic molecule using low pH, acetic acid or trifluoroacetic acid is added to the purified HSP-antigenic molecule complex to give a final concentration of 10% (vol/vol) and the mixture incubated at room temperature or in a boiling water bath or any temperature in between, for 10 minutes. The high and low molecular weight fractions are separated, for example, using centrifugation through a Centricon® 10 assembly (Millipore). The high and low molecular weight fractions are recovered. The remaining large molecular weight HSP70-peptide complexes can be reincubated in low pH to remove any remaining peptides. The resulting higher molecular weight fractions containing HSP are pooled and concentrated.

A method for the purification of uncomplexed HSP70 follows:

The HSP70-peptide complex is purified as described above. Once the HSP70-peptide complex is purified, the peptide may be eluted from the HSP70 by either of the following two methods. More preferably, the HSP70-peptide complex is incubated in the presence of ATP. Alternatively, the HSP70-peptide complex is incubated in a low pH buffer.

Briefly, the HSP-70 peptide complex is centrifuged through a Centricon® 10 assembly to remove any low molecular weight material loosely associated with the complex. The large molecular weight fraction can be removed and analyzed by SDS-PAGE while the low molecular weight can be analyzed by HPLC. In the ATP incubation protocol, the stress protein-peptide complex in the large molecular weight fraction is incubated with 10 mM ATP for 30 minutes at room temperature.

The resulting samples are centrifuged through a Centricon® 10 assembly. The high and low molecular weight fractions are recovered. The remaining large molecular weight HSP70-peptide complexes can be reincubated with ATP to remove any remaining peptides.

The resulting higher molecular weight fractions containing HSP70 are pooled and concentrated.

Many genes encoding HSPs have been cloned and sequenced, including, for example, human HSP70 (GenBank Accession Nos. M11717 (SEQ ID NO:1)); human HSP90 (GenBank Accession No. X15183 (SEQ ID NO:2)); human gp96 (GenBank Accession No. M33716 (SEQ ID NO:3) and X15187 (SEQ ID NO:4)); human HSP40 (GenBank Accession No. D49547 (SEQ ID NO: 5)); human BiP (Genebank Accession No. M19645 (SEQ ID NO:6)); mouse gp 96 (GenBank Accession No. M16370 (SEQ ID NO: 7); mouse BiP (Genebank Accession No. U16277 (SEQ ID NO:8)); and mouse HSP70 (GenBank Accession No. M35021 (SEQ ID NO:9)).

Other HSPs include HSP47 (described, for example, in WO 2000/010603, incorporated herein by reference), HSP65, HSP60, HSP65, HSP105, HSP105, and HSP110 (as described in U.S. Pat. Nos. 5,747,332 and 5,747,332, incorporated herein by reference).

The HSPs, alone or complexed to antigenic molecules, can be produced by recombinant DNA technology using techniques well known in the art. Methods which are well known to those skilled in the art can be used to construct expression vectors containing HSP coding sequences and/or antigenic molecule coding sequences and appropriate transcriptional/translational control signals. The coding sequence for the HSP is operatively linked to the regulatory elements necessary for expression of the nucleic acid molecule. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns (if introns are present), maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter. By “promoter” is meant a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included. Suitable methods for recombinant expression include, for example, in vitro recombinant DNA techniques, synthetic techniques and in vivo recombination/genetic recombination.

A variety of host-expression vector systems can be utilized to express the HSP genes. These include but are not limited to microorganisms such as bacteria (e.g., E. coli, Bacillus subtilis) transformed with recombinant bacteriophage DNA, plasmid DNA or cosmid DNA expression vectors containing the HSP coding sequence; yeast (e.g., Saccharomyces, Pichia) transformed with recombinant yeast expression vectors containing the HSP coding sequence; insect cell systems infected with recombinant virus expression vectors (e.g., baculovirus) containing the HSP coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the HSP coding sequence; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, for example, vectors which direct the expression of high levels of fusion protein products that are readily purified may be desirable. Such vectors include, but are not limited, to the E. coli expression vector pUR278 (accession number L09146), in which the HSP coding sequence can be ligated individually into the vector in frame with the lac Z coding region so that a fusion protein is produced; pIN vectors; and the like. pGEX vectors can also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned HSP gene protein can be released from the GST moiety.

In one embodiment, an insect system, Autographa californica nuclear polyhedrosis virus (AcNPV) is used as a vector to express foreign genes. The virus grows in Spodoptera frugiperda cells. The HSP gene can be cloned individually into non-essential regions (for example the polyhedrin gene) of the virus and placed under control of an AcNPV promoter (for example the polyhedrin promoter). Successful insertion of the HSP coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted gene is expressed. (U.S. Pat. No. 4,215,051; incorporated herein by reference).

In mammalian host cells, a number of viral-based expression systems may be utilized. In cases where an adenovirus is used as an expression vector, the HSP coding sequence can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing HSPs in infected hosts. Specific initiation signals may also be required for efficient translation of inserted HSP coding sequence. These signals include the ATG initiation codon and adjacent sequences. The efficiency of expression can be enhanced by the inclusion of appropriate transcription enhancer elements, transcription terminators, and the like.

In general, any type of mammalian expression vector can be used, although those with the highest transfection and expression efficiencies are preferred to maximize the levels of expression. Specific types of vectors which can be employed include herpes simplex viral based vectors such as pHSV 1; recombinant retroviral vectors such as MFG Moloney-based retroviral vectors including LN, LNSX, LNCX, and LXSN; vaccinia viral vectors including KVA; recombinant adenovirus vectors such as pJM17; second generation adenovirus vectors such as DE1/DE4 adenoviral vectors; and Adeno-associated viral vectors such as AAV/Neo. Specific suitable expression systems for this purpose include pcDNA3 (Invitrogen), plasmid AH5 (which contains the SV40 origin and the adenovirus major late promoter), pRC/CMV (Invitrogen), pCMU II, pZip-Neo SV, and pSRα (DNAX, Palo Alto, Calif.).

In addition, a host cell strain can be chosen which modulates the expression of the inserted sequences, or modifies and processes the HSP in the specific fashion desired. For example, choosing a system that allows for appropriate glycosylation is especially important in the case of gp96. Different host cells have characteristic and specific mechanisms for the post-translational processing and modification of proteins such as glycosylation. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be employed. Such mammalian host cells include but are not limited to CHO, VERO, BHK, HeLa, COS, MDCK, 293, 3T3, WI38, and the like.

In one embodiment of recombinant expression of HSPs, the histidine-nickel (his-Ni) tag system is used. In the his-Ni system, the HSP is expressed in human cell lines as a fusion protein which can be readily purified in a non-denatured form. In this system, for example, the gene of interest (i.e., the HSP gene) is subcloned into a vaccinia recombination plasmid such that the gene's open reading frame is translationally fused to an amino- or carboxy-terminal tag consisting of six histidine residues. Extracts from cells infected with recombinant vaccinia virus are loaded onto Ni²⁺ nitriloacetic acid-agarose columns and histidine-tagged proteins are selectively eluted with imidazole-containing buffers.

Kits for expressing isolating proteins using the his-Ni system are commercially available from Invitrogen, San Diego, Calif.

The uncomplexed HSPs promote pathogen elimination from a vertebrate organism, wherein the vertebrate organism has an infection caused by the pathogen, has been exposed to the pathogen, or is suspected of exposure to the pathogen. The HSPs can also be administered prophylactically to a vertebrate organism at risk of future exposure to the pathogen. In one embodiment, the vertebrate organism has an infection. As used herein, an infection is the detrimental colonization of a host organism by a pathogen. During infection, the pathogen utilizes the host's resources in order to multiply (usually at the expense of the host). The pathogen interferes with the normal functioning of the host and can lead to chronic wounds, gangrene, loss of an infected limb, and even death. The host's response to infection is inflammation. Pathogens include, for example, bacteria, parasites, fungi, and viruses.

In another embodiment, the vertebrate organism been exposed to the pathogen or is suspected of exposure to the pathogen. As used herein, exposure means contact with a pathogen by swallowing, breathing, or direct contact (such as through the skin or eyes). Exposure may be short term (acute) or long term (chronic). Acute exposure means a single exposure to a pathogen that may result in severe biological harm or death. In general, acute exposure lasts less than a day. Chronic exposure to a pathogen means exposure that occurs over an extended period of time, for example months or years. Post-exposure prophylaxis (PEP) is a prophylactic treatment started immediately after exposure or suspected exposure to a pathogen (such as a disease-causing virus), in order to prevent an infection caused by the pathogen from breaking out.

In another embodiment, the vertebrate organism has not been exposed to the pathogen and is treated as a form of pre-exposure prophylaxis. Pre-exposure prophylaxis (PrEP) is the long-term use of a prophylactic treatment for a disease prior to exposure to the cause of that disease, so that the prophylactic treatment will already be in place when exposure occurs. PrEP may either be able to prevent the disease from being contracted, or at least ensure that the resulting disease is treated from its outset, whether or not the patient is aware that infection has occurred.

Pathogens suitable for treatment by uncomplexed HSPs include, for example, viruses including hepatitis type A, hepatitis type B, hepatitis type C, influenza, varicella, adenovirus, HSV-1, HSV-2, rinderpest rhinovirous, echovirus, rotavirus, respiratory synctial virus, papilloma virus, papova virus, cytomegalovirus, echinovirus, arbovirus, huntavirus, coxsachie virus, mumps virus, measles virus, rubella virus, polio virus, HIV-1, and HIV-2; bacteria such as, Mycobacteria, Rickettsia, Mycoplasma, Neisseria and Legionella; intracellular protozoa, including, but not limited to, Leishmania, Kokzidioa, and Trypanosoma; intracellular parasites including, but not limited to, Chlamydia and Rickettsia; and fungi such as Absidia spp., Ajellomyces spp., Arthroderma spp., Aspergillus spp., Blastomyces spp., Candida spp., Cladophialophora spp., Coccidioides spp., Cryptococcus spp., Cunninghamella spp., Epidermophyton spp., Exophiala spp., Filobasidiella spp., Fonsecaea spp., Fusarium spp., Geotrichum spp., Histoplasma spp., Hortaea spp., Issatschenkia spp., Madurella spp., Malassezia spp., Microsporum spp., Mucor spp., Nectria spp., Paecilomyces spp., Paracoccidioides spp., Penicillium spp., Pichia spp., Pneumocystis spp., Pseudallescheria spp., Rhizopus spp., Rhodotorula spp., Scedosporium spp., Schizophyllum spp., Sporothrix spp., Trichophyton spp., and Trichosporon spp.

In one embodiment, the vertebrate organism has an infection caused by an NIAID (National Institute of Allergy and Infectious Diseases) category A, B, and C priority pathogen. NIAID category A pathogens include Bacillus anthracis (anthrax), Clostridium botulinum, Yersinia pestis, Variola major (smallpox), other pox viruses, Francisella tularensis (tularemia), and viral hemorrhagic fevers including those caused by arenaviruses (LCM, Junin virus, Machupo virus, Guanarito virus, Lassa fever), bunyaviruses (hantaviruses and Rift Valley fever), flavaviruses (Dengue), and filoviruses (ebola and marburg). NIAID category B pathogens include Burkholderia pseudomallei, Coxiella burnetii (Q fever), Brucella species (brucellosis), Burkholderia mailei (glanders), Ricin toxin (from Ricinus communis), Epsilon toxin of Clostridium perfringens, Staphylococcus enterotoxin B, Typhus fever (Rickettsia prowazekii), additional viral encephalitides (West Nile Virus, LaCrosse, California encephalitis, VEE, EEE, WEE, Japanese encephalitis virus, and Kyasanur Forest Virus), food and waterborne bacteria (Diarrheagenic E. coli, pathogenic Vibrios, Shigella species, Salmonella, Listeria monocytogenes, Camphylobacter jejuni, and Yersinia enterocolitica), food and waterborne viruses (Caliciviruses, Hepatitis A), and food and waterborne protozoa (Cryptosporidium parvum, Cyclospora cayatanensis, Giardia lamblia, Entamoeba histolytica, Toxoplasma, and Microsporidia). NIAID category C pathogens include tickborne hemorrhagic fever virus (Crimean-Congo Hemorrhagic fever virus), tickborne encephalitis viruses, yellow fever, multi-drug resistant TB, Influenza, other rickettsias, rabies, acute respiratory syndrome-associated coronavirus (SARS-CoV), and antimicrobial resistance excluding sexually-transmitted organisms.

In one embodiment, the vertebrate organism has an unidentified infection, that is, an infection wherein the causative pathogen is unidentified. Because the pathogen is unidentified, it is extremely difficult to treat such an infection using traditional therapies. An advantage of uncomplexed HSPs is that the phagocytic function of macrophages can be activated without identification of the pathogen.

In another embodiment, the infection is an emergent infection. Emerging infectious diseases are defined by the NIAID as diseases that have newly appeared in a population, or diseases that have existed in the past, but are rapidly increasing in incidence or geographic range. Re-emergence may also occur because of breakdowns in public health measures for previously controlled infections. Emerging diseases include, for example, AIDS, SARS, Lyme disease, Escherichia coli O157:H7 (E. coli), hantavirus, and others. Re-emerging diseases include, for example, malaria, tuberculosis, cholera, pertussis, influenza, pneumococcal disease, gonorrhea, and others. Additionally, there is the potential for diseases to emerge as a result of deliberate introduction into human, animal, or plant populations for terrorist purposes. Of pathogens causing emerging infectious diseases, 75% are zoonotic (able to transmit from animals to humans), with wildlife being an increasingly important source.

In one embodiment, the infection is an antibiotic-resistant infection. Antibiotic resistance is the ability of a pathogen to withstand the effects of an antibiotic. An uncomplexed HSP can be used to treat a variety of internal and external infections in subjects caused by antibiotic-resistant strains of Escherichia spp. such as E coli; Salmonella spp. such as Pasteurella spp.; Staphyloccocus spp.; Streptoccocus spp.; Corinebacterium spp.; Bacillus spp., such as Bacillus anthracis; Clostridium spp.; Spherophorus spp.; Candida spp.; Trychophyton spp.; Microsporum spp.; Micobacterium spp.; Yibrio spp.; Cryptosporidia spp.; Microsporidia spp.; Listeria monocytogenes; Lawsonia intracellularis; Treponema desynteriae; Enteroccocus spp.; Heamophylus spp.; Campylobacter spp.; Chlamydia spp.; Brucella spp., and other antibiotic-resistant bacterial species.

Antibiotic-resistant bacteria include penicillin-resistant Staphylococcus aureus; methicillin-resistant Staphylococcus aureus (MRSA); tetracycline-resistant Staphylococcus aureus; erthromycin-resistant Staphylococcus aureus; vancomycin-resistant Staphylococcus aureus (VRSA) (also termed GISA (glycopeptide intermediate Staphylococcus aureus) or VISA (vancomycin insensitive Staphylococcus aureus); linezoid-resistant Staphylococcus aureus; Enterococcus faecium; penicillin-resistant enterococcus; vancomycin-resistant enterococci (VRE); linezolid-resistant enterococcus (LRE); penicillin-resistant Streptococcus pneumoniae; penicillin-resistant gonorrhea; fluoroquinolone-resistant strains of E. coli; isoniazid-resistant Mycobacterium tuberculosis; rifampin-resistant Mycobacterium tuberculosis; coagulase-negative staphylococci (CNS); and combinations comprising one or more of the foregoing antibiotic-resistant bacteria.

Pseudomonas aeruginosa is another problematic pathogen that is difficult to treat because of its resistance to antibiotics. It is often acquired in the hospital and causes severe respiratory tract infections. P. aeruginosa is also associated with high mortality in patients with cystic fibrosis, severe burns, and in AIDS patients who are immunosuppressed. The clinical problems associated with this pathogen are many, as it is notorious for its resistance to antibiotics due to the permeability barrier afforded by its outer membrane lipopolysaccharide (LPS). The tendency of P. aeruginosa to colonize surfaces in a biofilm phenotype makes the cells impervious to therapeutic concentrations of antibiotics.

In another embodiment, the pathogen is an agent that is difficult to detect. Pathogens are typically identified by inoculation and incubation of growth media in the laboratory. Some pathogens, however, cannot readily be cultivated in vitro and are thus inherently difficult to identify. Although genetic sequencing can be employed, not all pathogens are readily identifiable from sequence information. Pathogens that are difficult to identify include, for example, pathogens engineered as biological weapons.

In one embodiment, the infection is an invasive fungal infection. Some of the most common pathogens associated with invasive fungal infections are the opportunistic yeasts, such as Candida spp. and Aspergillus spp. Thousands of Candida spp. cells can be present in an individual, primarily in the gastrointestinal tract, as a harmless commensal organism. However, Candida spp., such as C. albicans, cause opportunistic fungal infections. Infections can be localized, such as a vaginal infection or an oral infection, both of which cause a considerable degree of discomfort. In patients whose immune system is severely compromised (for example, prematurely born infants, patients infected with HIV, patients with hematological disease or cancer, and burn patients), the yeast can turn into a deadly pathogen causing systemic infections. Aspergillus spp., such as A. niger, are also opportunistic fungi which under certain conditions lead to infection, e.g., aspergillosis. The widespread use of azoles (e.g., triazoles and imidazoles such as ketoconazole, itraconazole, and fluconazole) that inhibit fungal membrane-sterol biosynthesis is causing the emergence of clinically-resistant strains of Candida spp.

In another embodiment, the infection is a systemic infection. Systemic infection is a generic term for infection caused by a pathogen, where the pathogen has spread actively or passively in the host's anatomy and is disseminated throughout several organs in different systems of the host. The infection can be disseminated in the digestive system, respiratory system, circulatory system, or other systems.

Examples of systemic infections include those due to blood born bacteria (septicemia), the pulmonary condition known as acute (or adult) respiratory distress syndrome (ARDS), and peritoneal infections. Sepsis syndrome and shock are triggered by the interactions of various microbial products in the blood, in particular, gram-negative endotoxins, with host mediator systems. In ARDS, lung spaces fill with fluid, impeding gas exchange and producing respiratory failure. The most common causes of ARDS are infection, aspiration, smoke and toxin inhalation, as well as systemic processes initiated outside the lung, including bacterial septicemia. Causative agents of peritoneal infections include Staphylococcus aureus, although many peritoneal infections are multimicrobial infections.

In another embodiment, the uncomplexed HSPs are also useful in the treatment of deep tissue infections such as those in infected body cavities and deep-rooted tissue infections. Staphylococcus aureus, for example, is an important human and animal pathogen that causes superficial, deep-skin, soft-tissue infection, endocarditis, and bacteremia with metastatic abscess formation and a variety of toxin-mediated diseases including gastroenteritis, scalded-skin syndrome and toxic shock syndrome.

In one embodiment, the uncomplexed HSPs are used to treat a wound site, such as, for example, an infected wound site. Infection of wounds by bacteria delays the healing process, since bacteria compete for nutrients and oxygen with macrophages and fibroblasts, whose activities are essential for the healing of the wound. Infection results when bacteria achieve dominance over the systemic and local factors of host resistance. Infection is therefore a manifestation of a disturbed host/bacteria equilibrium in favor of the invading bacteria. This elicits a systemic septic response, and also inhibits the multiple processes involved in wound healing. Lastly, infection can result in a prolonged inflammatory phase and thus slow healing, or may cause further necrosis of the wound. The granulation phase of the healing process will begin only after the infection has subsided.

Chronically contaminated wounds all contain tissue bacterial flora. These bacteria may be indigenous to the patient or might be exogenous to the wound. Closure or eventual healing of the wound is often based on a physician's ability to control the level of the bacterial flora. If clinicians could respond to wound infection as early as possible, this would lead to less clinical intervention/hospitalization and would reduce the use of antibiotics and other complications of infection. Early treatment with uncomplexed HSPs, which does not require identification of the contaminating pathogens, would be extremely useful in this situation.

Current methods used to identify bacterial infection rely mainly on judgment of the odor and appearance of a wound. With experience, it is possible to identify an infection in a wound by certain chemical signs such as redness or pain. Some clinicians take swabs that are then cultured in the laboratory to identify specific organisms, but this technique takes time.

Chronic wounds also lead to the formation of necrotic tissue, which in turn lead to growth of microbes. Debridement of necrotic tissue is deemed as an important wound bed preparation for successful wound healing. Sharp and surgical debridement rapidly remove necrotic tissue and reduce the bacterial burden, but also carry the greatest risk of damage to viable tissue and require high levels of technical skill. Chemical, mechanical and autolytic debridement are frequently regarded as safer options, although the risk to the patient of ongoing wound complications is greater. Thus, treatment of chronic wounds, including those comprising necrotic tissue, with uncomplexed HSPs would be extremely useful in this situation.

Another wound for which treatment with uncomplexed HSPs are beneficial is chronic diabetic ulcers. Topical agents may sometimes be applied over the infected region, however, topical anti-infective agents do not penetrate deep within the skin where a significant portion of the bacteria often reside. In addition, despite recent advances in chronic wound care, many lower extremity ulcers do not heal. Chronic ulcers of the lower extremities are a significant public health problem. Besides the large financial burden placed on the health care system for their treatment, they cause a heavy toll in human suffering. As the population ages and with the current obesity crisis in North America, venous, diabetic, and pressure ulcers are likely to become ever more common.

In yet another embodiment, HSPs are used to treat vertebrate organisms that have been exposed to or are suspected of exposure to particulate agents such as particulate pollutants. A pollutant is a contaminant that adversely alters the physical, chemical or biological properties of the environment. Particulate agents such as pollutants generally have a diameter of about 1 to about 20 microns. Vertebrate organisms can be exposed to particulate agents by, for example, inhalation, ingestion, or through the skin such as through an injury. An advantage of the use of uncomplexed HSPs is that particulate agent need not be identified prior to treatment.

Occupational lung diseases, for example, are caused by harmful particles that pass the nose or large airways and become trapped in the lungs. In the lungs, particles may dissolve and may be absorbed into the bloodstream. The solid particles that do not dissolve may be removed by the body's defenses. Particulate pollutants include, for example, asbestos, cadmium, mercury, silica, coal dust, carbon, tin oxide, beryllium, stone dust, graphite, and combinations comprising one or more of the foregoing pollutants. An advantage of the use of uncomplexed HSPs is that the phagocytosis process stimulated by the HSPs is nonspecific and can be used to treat exposure even when the particular environmental pollutant is unknown.

Particulate pollutants can cause severe health problems, particularly when inhaled. Exposure to pollutants such as asbestos, silica and metal dusts can, for example, lead to chronic pulmonary inflammatory diseases. Deposition of silica particles in the lung leads to silicosis, a disease of progressive respiratory failure caused by a fibrotic reaction. Exposure to asbestos leads to asbestosis, and mesothelioma (cancer of the mesothelial lining of the lungs and the chest cavity, the peritoneum or the pericardium).

The HSPs can also be employed to treat infection, exposure, or suspected exposure to a particulate agent comprising a biological agent. The uncomplexed HSPs arethus also useful in bioterrorism and biological warfare applications. Bioterrorism is terrorism using germ warfare, an intentional human release of a naturally-occurring or human-modified toxin or biological agent. Biological warfare, also known as germ warfare, is the use of a pathogen (e.g, bacteria or virus) or toxin as a weapon of war. Biological weapons are typically highly infective, potent, difficult to treat, and suitable for aerosol delivery. For example, anthrax is considered an effective biological weapon for several reasons. First, it forms hardy spores, perfect for dispersal aerosols. Second, pneumonic infections of anthrax usually do not cause secondary infections in other people, confining the agent to the target. A mass attack using a particulate agent typically requires the creation of aerosol particles of 1.5 to 5 micrometres. Thus, when employed as biological weapons, biological agents often comprise a carrier.

Diseases considered for weaponization, or known to be weaponized include anthrax, Ebola, Bubonic Plague, Cholera, Tularemia, Brucellosis, Q fever, Machupo, Coccidioides mycosis, Glanders, Melioidosis, Shigella, Rocky Mountain Spotted Fever, Typhus, Psittacosis, Yellow Fever, Japanese B Encephalitis, Rift Valley Fever, and Smallpox. Naturally-occurring toxins that can be used as weapons include Ricin, SEB, Botulism toxin, Saxitoxin, and many Mycotoxins. The organisms causing these diseases are known as select agents. Their possession, use, and transfer are regulated by the Centers for Disease Control and Prevention's Select Agent Program.

HSPs and HSP-antigenic molecule complexes are administered to vertebrate organisms such as mammals and birds. Exemplary mammalian subjects include primates, dogs, cats, mice, rats, horses, cows, pigs, and the like, preferably humans, in doses of about 1 μg to about 5000 μg, preferably of about 1 μg to about 1500 μg. In mammals, about 30 μg to about 500 μg, preferably intradermally, with about 5 μg to about 100 μg intradermally even more preferred. An effective dose for promotion of wound healing in a mouse model is 30 μg gp96 administered intradermally for mice of average mass of 20-25 g.

Methods of introduction include, but are not limited to, intradermal, intramuscular, intraperitoneal, intravenous, subcutaneous, intranasal, epidural, and oral routes. The HSPs or complexes may be administered by a convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local; this may be achieved, for example and not by way of limitation, by topical application, by injection, by means of a catheter, by means of a suppository, or by means of an implant, said implant being of a porous, non-porous, or gelatinous material, including membranes, such as sialastic membranes, or fibers.

In a specific embodiment, the HSP compositions are administered, either intradermally or subcutaneously, with sites of administration varied sequentially. For example, and not by way of limitation, the doses recited above are given once weekly for a period of about 4 to 6 weeks, and the mode of administration is varied with each administration. Each site of administration may be varied sequentially. Thus, by way of example and not limitation, the injections can be given, either intradermally or subcutaneously, locally (i.e., near a wound site or site of infection) or at a site distant from the site of damage. The same site can be repeated after a gap of one or more injections. Also, split injections can be given. Thus, for example, half the dose can be given in one site and the other half in another site on the same day.

After 4-6 weeks, further injections are preferably given at two-week intervals over a period of time of one month. Later injections can be given monthly. The pace of later injections can be modified, depending upon the patient's clinical progress and responsiveness to the therapy. Alternatively, the mode of administration is sequentially varied, e.g., weekly administrations are given in sequence intradermally or subcutaneously.

The uncomplexed HSPs or HSPs complexed with antigenic molecules, can be formulated into pharmaceutical preparations for administration to mammals, preferably humans, for promotion of phagocytosis at a site of infection or a wound site. Compositions comprising an HSP formulated in a compatible pharmaceutical carrier can be prepared, packaged, and labeled for treatment of infection.

If the complex is water-soluble, then it can be formulated in an appropriate buffer, for example, phosphate buffered saline or other physiologically compatible solutions. Alternatively, if the resulting complex has poor solubility in aqueous solvents, then it can be formulated with a non-ionic surfactant such as Tween, or polyethylene glycol. Thus, the compounds and their physiologically acceptable solvates can be formulated for administration by inhalation or insufflation (either through the mouth or the nose) or oral, buccal, parenteral, rectal administration.

For oral administration, the pharmaceutical preparation can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Preparations for oral administration can be suitably formulated to give controlled release of the active compound.

For buccal administration, the compositions can take the form of tablets or lozenges formulated in conventional manner.

For administration by inhalation, the HSP compositions are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The HSP compositions can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The HSP compositions can be formulated into creams, lotions, ointments or tinctures, e.g., containing conventional bases, such as hydrocarbons, petrolatum, lanolin, waxes, glycerin, or alcohol. The compositions can also be formulated in rectal compositions such as suppositories or retention enemas, e.g., containing conventional suppository bases such as cocoa butter or other glycerides.

In addition to the formulations described previously, the HSP compositions can also be formulated as a depot preparation. Such long acting formulations can be administered by implantation (e.g., subcutaneously or intramuscularly) or by intramuscular injection. Thus, for example, the HSP compositions can be formulated with suitable polymeric or hydrophobic materials (e.g., as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

The HSP compositions can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

The invention also provides kits for carrying out the therapeutic regimens of the invention. Such kits comprise in one or more containers therapeutically effective amounts of the HSP or HSP-antigenic molecule complexes in pharmaceutically acceptable form. The HSP or HSP-antigenic molecule complex in a vial of a kit can be in the form of a pharmaceutically acceptable solution, e.g., in combination with sterile saline, dextrose solution, or buffered solution, or other pharmaceutically acceptable sterile fluid. Alternatively, the complex can be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit further comprises a needle or syringe, preferably packaged in sterile form, for injecting the complex, and/or a packaged alcohol pad. Instructions are optionally included for administration of HSP or HSP-antigenic molecule complexes by a clinician or by the patient.

The invention is further illustrated by the following non-limiting examples:

### EXAMPLES

Materials and Methods

Cell Culture. All cell lines were obtained from ATCC. RAW 264.7, RAW 309 and J774A.1 were maintained in Dulbecco's modified Eagle's medium (DMEM) (Gibco, Invitrogen Corporation) with 10% heat inactivated fetal calf serum (FCS) and RAW264.7 NO- was maintained in Roswell Park Memorial Institute (RPMI)-1640 (ATCC) with 10% heat inactivated FCS at 37° C., 5% CO₂.

Mice. I-A^{d}-restricted DO11.10 T cell receptor (TCR)-αβ-transgenic and C57/BL6J mice (6-8 wk old) were purchased from The Jackson Laboratory (Bar Harbor, Me.). Mice were housed in the vivarium of the Center of Laboratory Animal Care in University of Connecticut Health Center.

Reagents. All chemicals, unless otherwise specified were purchased from Sigma (St. Louis, Mo.) and all sterile non-charged plastic ware from Corning Inc. (Corning N.Y.). Alexa Fluor® 488-labeled Saccharomyces cerevisiae, Escherichia coli, fluorescent and unlabeled polystyrene microspheres (size 3 μm) were purchased from Molecular Probes and Sigma respectively. Anti-HSP70 antibodies were obtained from Stressgen.

Heat Shock Proteins (HSPs). HSPs (HSP70, HSP90 and GP96) were purified from murine livers as described earlier. HSPs were prepared as a complex with endogenous peptide, except when using adenosine triphosphate (ATP)-treated HSP70 that was utilized to assess the role of peptides in phagocytosis. ATP-treated HSP70 removes all the peptides associate with HSP70 itself, whereas the purification of HSP70 using adenosine diphosphate ADP does not. Purity was established by SDS-Page electrophoresis and immunoblotting and HSPs were quantified using Bradford analysis. LPS content was measured by the Limulus Amebocyte Lysate (LAL) assay (LAL kit QCL-1000; Biowhittaker, Walkersville, Md.).

Phagocytosis Assay in vitro. RAW 264.7, RAW264.7 (NO-) and J774A.1 cells were cultured in DMEM or RPMI (RAW NO-) with 10% FCS, harvested, and washed. 3×10⁵ cells/well were incubated in serum-free Earle's minimal essential medium (EMEM) in a 24-well plate for 1 hour at 37° C. Alexa Fluor® 488-labeled particles (inert polystyrene microspheres; or yeast, Saccharomyces cerevisiae or Gram-negative bacteria, Escherichia coli, 40 particles/macrophage) were added and co-incubated in a dark environment for 60 minutes in serum free EMEM with or without HSPs, LPS, or control proteins. At the end of 60 minutes incubation, the plate was covered in a dark container on ice. Specific measures were taken to exclude the fluorescence that resulted from particles that were outside of the cell or sticking to the surface of the cell. This was done by using trypan blue, which quenches all the fluorescence outside of the cell but does not quench the internalized particles. Specifically, trypan blue (0.8 mg/ml) was added for 60 seconds and plates were analyzed immediately using FluorImager SI (Molecular Dynamics/Amersham). Since the emission of Alexa Fluor® 488, about 519 nm at an excitation of about 490 nm, falls within the absorbance range of trypan blue (475-675 nm), the fluorescence of the sample only represents the intracellular fluorescence. External or surface-bound fluorescence is quenched. Using Student's t test, the results were analyzed and P-values were calculated.

Purification of lipid rafts. Lipid rafts were purified as DRMs (detergent resistant membrane) using non-ionic detergents following sucrose gradient centrifugation. The cells used for lipid raft purification were treated with or without 30 mM methyl-β-cyclodextrin (MCD) 30 minutes at 37° C., after washed with cold PBS incubated with HSP70-HRP (horseradish peroxidase) complexes (HRP conjugation kit—Alpha Diagnostic, San Antonio, Tex.) for 30 minutes on ice. After incubation, cells were washed 3× with PBS and lysed with 2 ml MES (4-morpholinoethanesulfonic acid) buffered saline (MBS; 150 mM NaCl, 20 mM MES, pH 6.5, 500 mM PMSF and 5 mM iodoacetamide) containing 0.5% Triton X-100 or 1% for 30 minutes on ice or 7 minutes at 37° C. respectively. The cells were mixed with an equal volume of 90% sucrose in MEB buffer (150 mM NaCl, 20 mM MES [pH 6.5]) and placed at the bottom of the centrifuge tube. The sample was overlaid with 5.5 ml of 30% sucrose and 4.5 ml of 5% sucrose in MBS and centrifuged at 100,000×g for 16 hours (SW28, Beckman Instruments, Palo Alto, Calif.). Fractions of 1 ml each were collected from the bottom of tube and each fraction was analyzed by SDS-PAGE and immunoblotting.

Proliferation of CD4+ T cells and INF-γ (interferon gamma) release. Unlabeled S. cerevisiae (80 μg) was incubated with excess chicken ovary albumin (OVA) at 37° C. for 1 hour and washed repeatedly to eliminate free OVA protein. S. cerevisiae-OVA complexes (OVA-coated yeast) were administered to macrophages treated with or without HSP70. Macrophages were then washed and irradiated at 12,000 rads and assessed for viability by trypan blue exclusion. Subsequently, they were co-cultured with CD4+ T lymphocytes that were purified from spleens of DO 11.10 mice and labeled with carboxyfluoroscein succinimidyl ester (CSFE). Purification of CD4+ T lymphocytes was performed using the (magnetic cell sorting) MACS purification columns and αCD4 microbeads (Miltenyi Biotech, CA). Following the 48-hour incubation, proliferation of CD4+ cells was determined with FACS and IFN-γ release in the supernatant was tested by enzyme-linked immunosorbent assay (ELISA; Pierce, Ill.).

Mice. Female BALB/cJ mice 6-8 weeks of age (Jackson Laboratories, Bar Harbor, Me.) were housed in the vivarium at the University of Connecticut Health Center, Farmington, Conn.

Surgery and Treatment. Mice were anesthetized using Ketamine (2.0 mg/mouse) and Xylazine (0.1 mg/mouse) were administered intraperitoneally (IP) and the dorsal skin was shaved. After sterile prepping of the area, a full thickness circular wound, 8 mm diameter, was made on the dorsal skin using a disposable biopsy punch (Miltex Inc, York, Pa.) on the midline of the dorsum approximately at the iliac crest. The mice were then randomly assigned into the treatment groups. Initial wound measurements were performed while the mice were still anesthetized. Mice were then treated on days 0, 2, 4, and 6 unless otherwise specified. All agents used as treatment were diluted in 0.5 ml of PBS and injected using a 25-gauge needle in the subcutaneous space in the nape of the neck. The surgical procedures, post-operative monitoring, and safety concerns were approved by the Institutional Animal Care and Usage Committee.

Measurements and Analysis. Wound healing of open wounds is a complex process with many parameters to consider. Measuring one parameter does not take into account all of these processes, however wound closure is an good indicator of overall wound healing and is widely used to assess wound healing. The wound surface area was determined by measuring the craniocaudal and lateromedial distances using vernier calipers in perpendicular axis on days 0, 2, 4, 6, 9, and 11 postoperatively unless noted. Changes in wound area were expressed as percent wound closure. Wound closure data was plotted as average percent wound closure ±SEM unless otherwise noted. All calculations and data analysis were performed using Microsoft Excel and Sigma Plot. Sigma Plot was used to perform Student's t-tests for reported P values.

Macrophage Cell Culture, Pre-treatment, and Adoptive Transfer. RAW 264.7 macrophages (ATCC) were grown as described by ATCC. Cells were harvested by gently scraping them from the cell culture flasks, centrifuged and washed once in cultured grade PBS. Cell viability was confirmed by trypan blue exclusion and 10⁷ cells were treated with either 30 μg/0.5 ml of HSP70, lipopolysaccharide (LPS), mouse serum albumin (MSA), or in PBS at 4° C. for 1 hour. They were then washed 3 times with PBS, and were re-suspended to achieve a concentration of 400,000, 40,000, or 4,000 cells per 0.5 ml PBS. Adoptive transfer of these cells was performed by subcutaneous injections in the nape of the neck using a 25-gauge needle in mice that were wounded as previously described. Adoptive transfers were given on days 0, 2 and 4 post-operatively.

In vitro Macrophage Phagocytosis of Wound Debris. Fresh wounds were harvested from female BALB/cJ mice 6-8 weeks of age. Wounds were cut into small pieces in PBS with PMSF and dounced on ice. Large debris was removed by straining the wound lysate through a 0.7 μM mesh strainer then centrifuged at 1,200 rpm for 10 minutes. The pellet was washed twice with PBS, labeled with Alexafluor488 following manufacture's protocol (Molecular Probes, Eugene, Oreg.). The labeled wound debris was administered to 3×10⁵ RAW 264.7 macrophages in serum-free medium in 24-well plates. Phagocytosis was performed either at 37° C. or 4° C. for 30 minutes after which 0.8 mg/ml Trypan blue was used to quench the fluorescence of the non-internalized wound debris. The plates analyzed with FluorImager plate reader.

Inhibition of Phagocytosis In vivo. Intravenous injection of gadolinium chloride inhibits phagocytosis of macrophages without affecting their viability or affecting other cell types. Mice were treated (on day minus-1 and day 0) with GdCl₃ (10 mg/kg body weight in 200 μl of distilled water) administered intravenously via the retro-orbital venous plexus. Subsequently the mice were wounded as previously described (Day 0) and treated with PBS and HSP70 (30 μg/dose) given subcutaneously on day 0.

Wound and Skin Lysate Cytokine Production. Mice were wounded and treated as stated previously. Wounds were harvested 24 hours after treatment, pulverized in 5 ml of PBS and large debris was removed by straining the wound lysate through a 0.2 μM mesh strainer. The soluble fraction was then centrifuged for 20 minutes at 14,000 rpm. Protein concentration was then assessed by Bradford assay. Skin samples were taken and processed in the same manner. 250 μg of each sample was then tested for the presence of monocyte chemotactic peptide-1 (MCP-1), tumor necrosis factor-α (TNF-α), and interleukin 6 (IL-6) using the BD Biosciences cytometric bead assay (CBA) mouse inflammatory kit (BD Biosciences Pharmingen, San Diego, and CA.) following the manufacture's protocol.

### Example 1

### HSPs Enhance Macrophage-Mediated Antigen Uptake

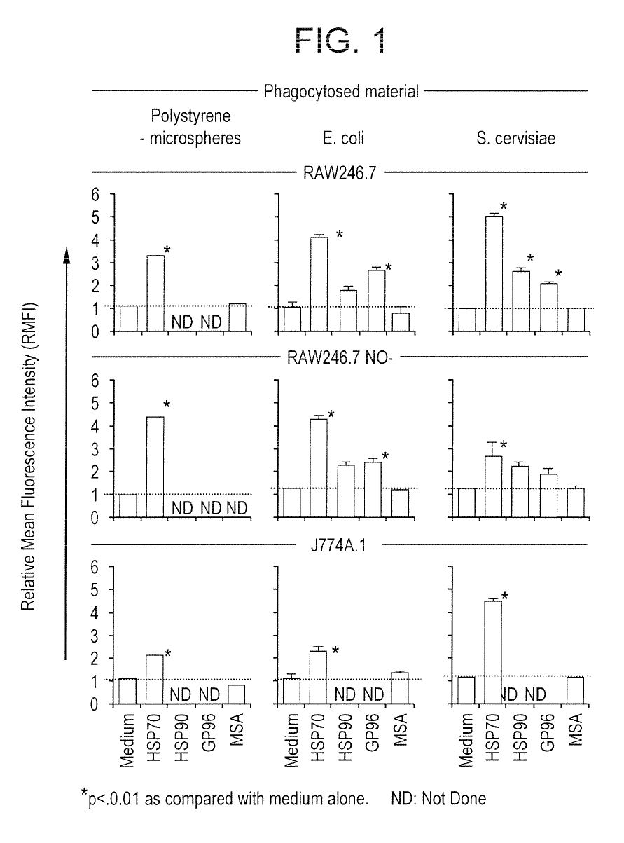

Murine macrophage lines (RAW264.7, J774.A1 or RAW264NO⁻) were treated with either one of the HSPs (HSP70, HSP90 or gp96) (100 μg/ml) or with non-HSP control proteins and co-incubated with either Alexa Fluor® 488-labeled inert polystyrene microspheres, yeast (Saccharomyces cerevisiae or Gram-negative bacteria (Escherichia coli (40 particles/macrophage). A phagocytosis assay was performed employing specific techniques to exclude external cell-surface binding of particles and measuring the actual fluorescence from internalized material alone. Macrophages treated with any of the three HSPs consistently showed an increase in uptake of microbial or non-microbial materials as compared to those treated with control proteins or with buffer (FIG. 1). HSP-mediated phagocytosis was quantified by measuring the internalized fluorescence measured by FluorImager SI and Relative Mean Fluorescence Intensity (RMFI) was compared between the treatment groups (as indicated). All the treatment groups were compared to medium alone using the Student's t test and significance denoted by P<0.01. The error bar represents one standard deviation. The results shown are a cumulative analysis of 3 experiments, 3 wells/group. The increase in uptake ranged from 2-6 times the basal rate and included the internalization of a variety of materials tested.

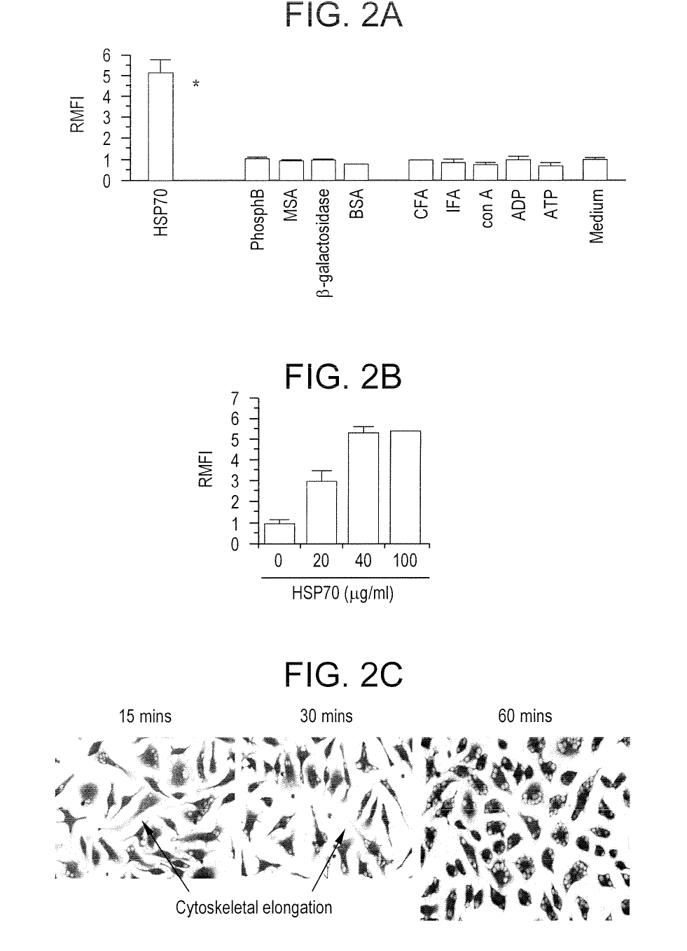

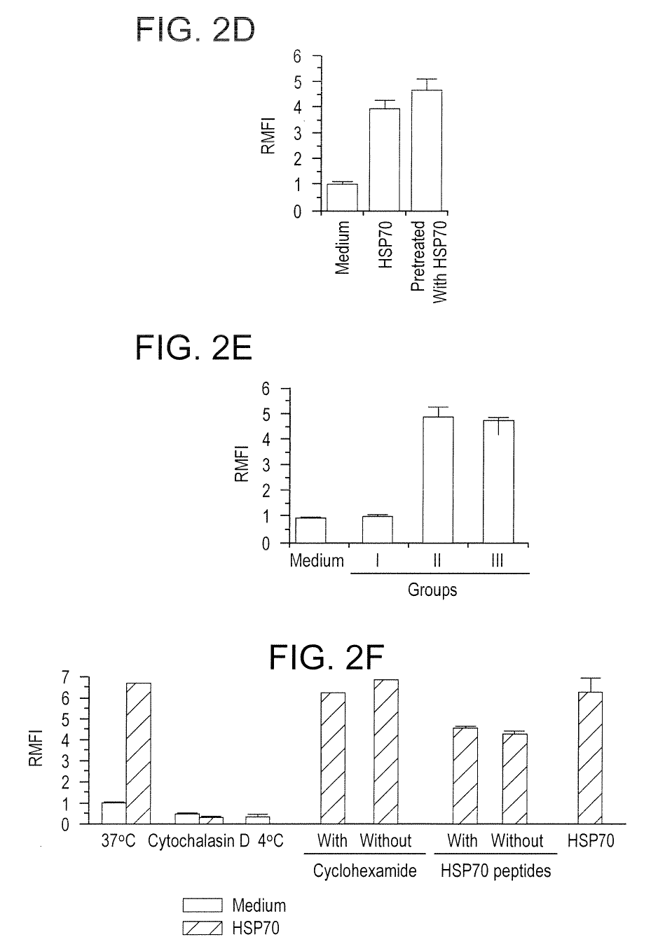

HSP70 was selected for further study because its molecular functions and its influence on various aspects of tissue protection are well-known. The specificity of HSP70-mediated antigen uptake was tested by treating macrophages with either HSP70 (100 μg/ml) or with equimolar amounts of HSP-controls including beta galactosidase; phosphorylase B; and MSA; (molecular weights of these proteins correspond to those of the HSPs tested); bovine serum albumin (BSA) (100 μg/ml); concavalin A (6 μg/ml); and Complete and Incomplete Freunds Adjuvant (10 μl/well); (potent stimulators of antigen presenting cell (APC)-function). None of control proteins used enhanced uptake as compared to treatment with HSP70. Further, reagents commonly used in HSP purification, including buffers, ADP (3 mM); and ATP (3 mM); were ineffective (FIG. 2A).

Increasing the doses of HSP70 (range 10-100 μg/ml) revealed that doses less than 20 μg/ml were unable to stimulate uptake and the peak effect of HSPs occurred at 40 μg/ml (FIG. 2B) and reached a plateau through 100 μg/ml. Doses up to 200 μg/ml did not result in any further increase in the quantity of phagocytosis. Physiologically, this dose is well within the range of the concentration of HSP70 that is observed from lysis of 1 gram of tissue lysis. Lysate from 1 gram of tissue typically contains 200 μg of HSP70. HSP70 in the dose of 100 μg/ml was employed to correspond to 0.5 grams of lysed tissue. Injury causing lysis of as little as 0.5 grams of tissue could potentially mimic the conditions used in vitro wherein macrophages are treated with 100 μg/ml concentration of HSP70. Viability of cells treated was confirmed using trypan blue exclusion to ensure that the HSP-treatment was not cytotoxic. Time-titration was performed by treating macrophages with HSPs or controls for increasing time-periods, ranging from 10-90 minutes. In the HSP-treated group, antigen uptake reached a plateau within 45 minutes. Time points beyond 90 minutes were not tested. Representative microphotographs of macrophages visualized at 10× magnification using haematoxylin and eosin (H&E) stain at 15, 30 and 60 minutes showed morphologic changes indicative of ongoing uptake, which ceased at 60 minutes (FIG. 2C). At 15 and 30 minutes the macrophages show ongoing phagocytic activity (elongated cells with cytoskeletal alterations) whereas at 60 minutes the cells are round, and appear quiescent.

In order to address whether HSP70 somehow coated the yeast particles thereby acting like an ‘opsonizing agent’, the following experiment were performed. First, macrophages were pre-treated with HSP70 and washed until no free HSPs remained in the wash. Then they were administered Alexa Fluor® 488-labeled yeast (S. cerevisiae). Pre-treating the macrophages with HSP prior to administering the yeast enhanced the uptake of yeast to the same extent as when both (HSP and yeast) were administered simultaneously (FIG. 2D). For comparison, macrophages were treated with HSP70 and administered the yeast at the same time.

Second, yeast particles were co-incubated with HSP70 (100 μg/80 μg of yeast, 37° C., 60 minutes) to create an HSP-coated-yeast complex. Unbound HSP70 was removed by washing with PBS. The presence of the HSP-coated yeast complex was confirmed by sodium dodecyl sulfate-polyacrylamide gel electrophoresis (SDS-PAGE) and immunoblotting with HSP70 antibodies. These complexes were then co-incubated with macrophages with the following experimental groups: Group I: HSP70-coated yeast; Group II: Yeast+HSP70; Group III: HSP70-treated macrophages+HSP70-coated yeast. As shown in FIG. 2E, macrophages that were treated with HSP70-coated yeast (Group I), did not demonstrate increased uptake of yeast as compared to Group II where both the HSP70 and yeast were administered simultaneously, indicating that HSPs do not act as opsonizing agents. Experimental groups (as indicated) were tested for their ability to enhance phagocytosis.

Macrophages treated with HSP70 at conditions that inhibit actin polymerization, i.e., at either 4° C. instead of 37° C. or in the presence of cytochalasin-D (4 μM), showed inhibition of the effects of HSP70 (FIG. 2F) indicating that HSP70-mediated internalization was via actin-dependent cytoskeletal rearrangement, essential characteristics of phagocytosis. The error bar represents one standard deviation. The results shown are a cumulative analysis of 3 experiments, 3 wells/group.

Macrophages treated with HSP70 either in presence or absence of cyclohexamide (1 mM; ribosomal protein synthesis inhibitor), showed similar increase in HSP-mediated phagocytic uptake as macrophages treated with HSP70 alone (FIG. 2F), indicating that synthesis of new intracellular proteins is not essential for HSP-mediated phagocytosis. Trypan blue exclusion was undertaken to confirm that the cells were viable through the cyclohexamide treatment.

Finally, macrophages treated with either ATP-purified HSP70 (ATP removes peptide from the HSP70) or with ADP-purified HSP70 (peptides remain bound to the HSP70) demonstrated the same degree of HSP-mediated phagocytosis (FIG. 2F) indicating that the HSP70-mediated effect was independent of the presence of chaperoned peptides.

### Example 2

### HSP70-Mediated Phagocytosis is not Due to Contamination by Endotoxin

It was previously shown that tumor necrosis factor alpha activation that occurred during treatment with HSP70 preparations was not due to HSP70, but instead due to the presence of contaminating lipopolysaccharide (LPS) in the preparations. LPS activates tumor necrosis factor alpha (TNF-α) via CD14 and Toll-like receptor-4 (TLR-4)-mediated signal transduction.

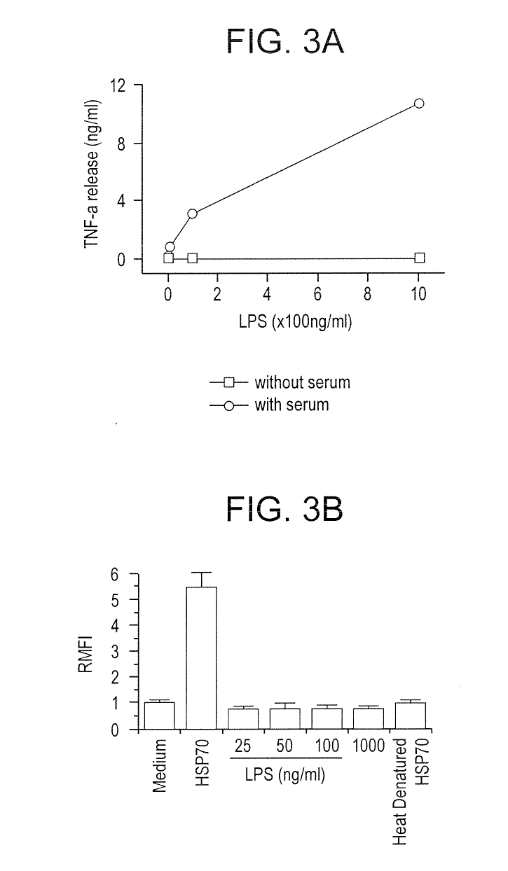

HSP70 used in all assays was purified using LPS-free sterile packaged plastic ware, baked glass ware (420° F. for 4 hours) and using endotoxin-free culture grade reagents to minimize inadvertent LPS contamination. Further, LPS levels and activity in the purified HSPs were quantified and confirmed to be <1 EU/mg of protein. Next, HSPs and LPS were compared for their influence on phagocytosis in presence or absence of serum. These conditions were specifically chosen since LPS-binding protein (LBP), an essential mediator for LPS function, is not present in serum-free conditions. As a control, macrophages were treated with LPS in presence or absence of serum and their ability to elicit TNF-α release was compared (FIG. 3A). Having established that serum-free conditions rendered LPS ineffective at stimulating macrophages, the same conditions (serum-free) were used to test the ability of HSP70 to stimulate macrophages. Briefly, macrophages were treated with HSP70 (100 μg/ml) or LPS (different doses ranging 25 ng-1 μg/ml) in serum-free conditions for 1 hour and a phagocytosis assay was performed. HSP70 was able to enhance phagocytosis equally whether in the presence or absence of serum whereas LPS was unable to enhance phagocytosis in serum-free conditions (FIG. 3B). The error bar is one standard deviation. The results shown are a cumulative analysis of 3 experiments, 3 wells/group. Since HSPs do not act via the LBP, their activity is not affected by the absence of serum.

Lastly, since heat denatures HSPs but not LPS, HSP70 preparations were heated at 100° C. for 20 minutes and tested for phagocytosis. Heat-denatured HSP70 samples lost the ability to enhance phagocytosis as compared to intact HSPs (FIG. 3B), indicating that the HSP70-mediated phagocytosis was due to intact HSP70 alone and not due to any contaminating LPS. Taken together, these measures indicate that contaminating LPS (if any) is not responsible for HSP-mediated phagocytosis.

### Example 3

### Exogenous HSP70 Binds the Lipid Raft (LR) Microdomain of the Macrophage Plasma Membrane

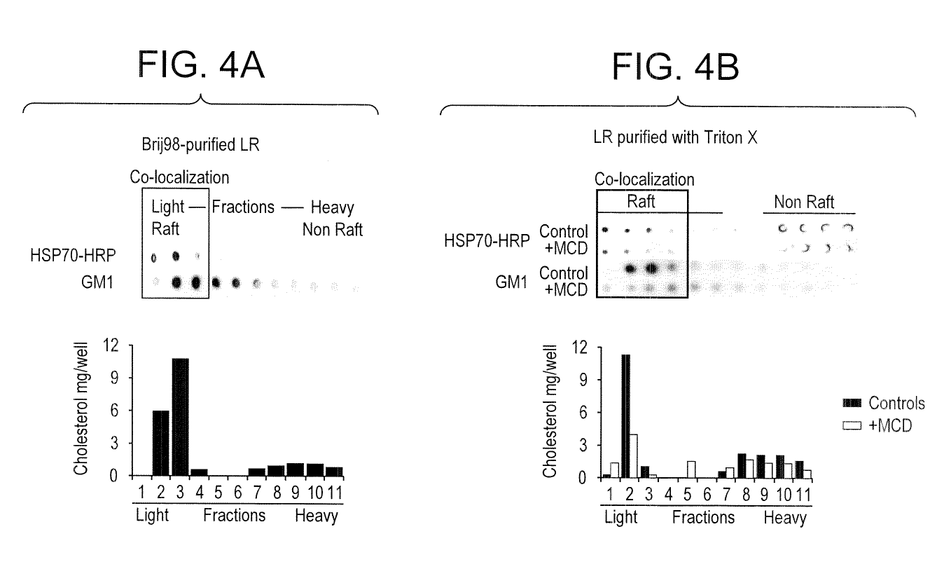

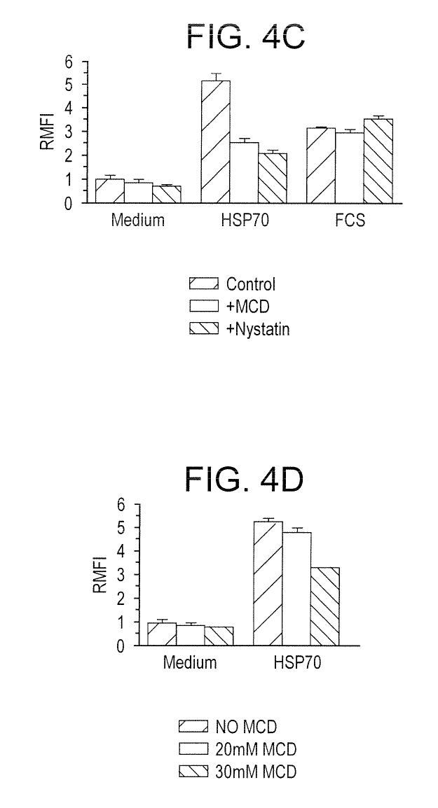

Briefly, RAW264.7 macrophages were treated with exogenous HRP-labeled HSP70 (100 μg/ml) or were left untreated for 60 minutes and washed with PBS three times to remove free HSPs. Subsequently, they were lysed in MES buffer and processed for purification of LRs. One of two different types of detergents was used: Brij98 or Triton X-100 to fractionate the material to isolate detergent resistant fraction called detergent resistant membranes (DRMs). This fraction was further purified into light and heavy components using a sucrose gradient. These fractions obtained (light: LR and heavy: non-LR) were then analyzed using immunoblotting to detect HRP activity of HSP70-HRP complexes or tested for the presence of an LR-associated ganglioside GM1 by using HRP-cholera toxin B. Similarly, these fractions were tested for the amount of cholesterol using (Biovision Research Products, CA). LR fractions were purified from HSP70-treated macrophages using Brij98 (FIG. 4A) or Triton X-100 (FIG. 4B). These methods showed that exogenously administered HSP70 and ganglioside GM1 (GM1), co-localized on the LR-microdomain of the macrophage plasma membrane binding (FIG. 4A). Further fractionation using a sucrose gradient into LR (light fractions) or non-LR (heavy fractions) microdomains was undertaken. These fractions were assayed for the following: HRP activity of HRP-HSP70 complexes (as indicated); the presence of GM1 by using HRP-cholera toxin-B (as indicated), and the amount of cholesterol. Similarly cholesterol levels of the fractions that bound exogenous HSP70 were higher. Collectively, these results indicated that HSP70 bound to the same fractions that were enriched in LR-associated molecules including GM1 and cholesterol and co-purified with the LR-microdomains as purified by using two separate detergents.

Further, macrophages were treated exogenous HRP-labeled HSP70 (100 μg/ml) in presence of methyl-β-cyclodextrin (MCD) (30 μM), which is known to disrupt the LR-integrity. Subsequently macrophages were processed for purification of LRs by using Triton X-100. As shown in FIG. 4B, there was a significant reduction in the binding of exogenously administered HSP70 when macrophages were treated with MCD. A similar reduction in the presence of GM1 and cholesterol further supported the evidence that this reduction in HSP70-binding was due to the disruption of the LR. In order to ensure that the viability of cells was not affected by treatment with MCD we performed a trypan blue exclusion that revealed a 95% viability in both MCD-treated or untreated cells.

Next, macrophages were treated with medium alone, HSP70 or fetal calf serum (FCS) in presence of either nystatin or MCD (30 μM) (both agents are known to disrupt the LR integrity. Further, the ability of macrophages to bind HSP70 was tested in the presence of LR-disrupting drug MCD. Macrophages were treated MCD (30 mM), washed with cold PBS then incubated with exogenous HRP-labeled HSP70 (100 g/ml) for 30 min on ice. Subsequently macrophages were processed for purification of LRs by using Triton X-100 and tested for the presence of HSP70; GM1; and assayed quantitatively for the amount of cholesterol (FIG. 4B). Subsequently the cells were administered Alexa Fluor® 488-labeled yeast (40 particles/macrophage) and amount of yeast internalized was quantified as described in Materials and Methods. As a control macrophages treated with serum were also included in the assay to test the effects the LR-disrupting drugs on Fc receptor γ (FcRγ)-mediated antigen uptake. Macrophages treated with FCS served as controls to assess the effects of LR-disrupting drugs on opsonic phagocytosis. The results shown are a cumulative analysis of 3 experiments, 3 wells/group. As shown in FIG. 4C, HSP70-mediated phagocytosis was inhibited in the presence of LR-disrupting drugs whereas opsonic phagocytosis via the FcRγ was not.

Further, macrophages were treated with HSP70 (100 μg/ml) in presence of varying doses of MCD (ranging from 0-30 mM). As seen in FIG. 4D, the inhibitory effect that MCD has on the macrophage response to HSP70 is titratable depending upon the dose of MCD used.

### Example 4

### Increased HSP70-Mediated Phagocytosis Enhances Major Histocompatibility Complex (MHC)-II Antigen Processing and Presentation

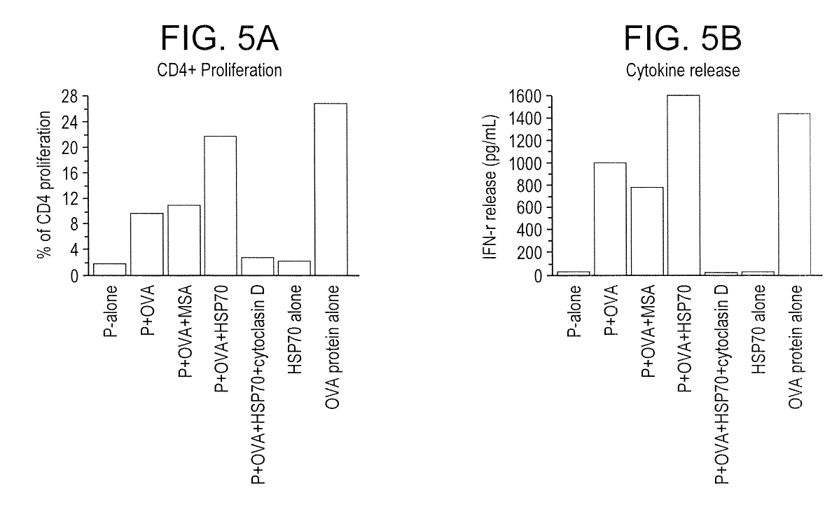

Alexafluor labeled S. cerevisiae were coated with whole ovalbumin (OVA) protein 1 mg/ml by co-incubation at 37° C. for 20 minutes and washed until free OVA protein was removed (washes were tested by gel electrophoresis using SDS-PAGE and immunoblotting). The association itself was confirmed by SDS-PAGE analysis and immunoblotting using antibody to OVA (Sigma, MO). The OVA protein-coated-yeast complexes (OVA-coated yeast) were administered to 4×10⁵ macrophages (40 yeast/macrophage) that were treated with either HSP70 (100 μg/ml) or control proteins in the presence or absence of cytochalasin-D to confirm that the process is phagocytosis-dependent (FIG. 5A). After 2 hours, the macrophages were washed, irradiated, to prevent macrophage-proliferation, and co-cultured with carboxyfluoroscein succinimidyl ester (CSFE)-labeled 2×10⁵ CD4+ T cells purified from spleens of DO 11.10 mice (1-A^{d}-restricted DO 11.10 T cell receptor (TCR)-αβ-transgenic mice). The DO 11.10 strain is transgenic against a MHC-II epitope of OVA protein and CD4+ T cells from these animals proliferate on being exposed to the antigenic epitope but not to the whole OVA protein. Resultant proliferation of the CSFE-labeled CD4+ was measured by FACS as an indirect measure of the amount of OVA protein processed and its peptide epitope presented in context of MHC-II. In order to quantify the effector function of the CD4+ cells, the production of interferon gamma (IFN-γ) by the CD4+ T cells was measured using ELISA. As shown in FIG. 5B HSP70-treated macrophages cause an increase in CD4+ proliferation to 20% as compared to those treated with medium alone (1.7%). Further, cytochalasin-D treatment abrogated the proliferation caused by HSP70-treatment indicating the net result was phagocytosis-dependent. The net increase in CD4+ cells in response to HSP70-treatment was concordant with the increase in their ability to produce IFN-γ as compared to treatment with ova-coated yeast alone or HSP70 given alone (FIG. 5C). Taken together these results indicated that HSP70-mediated a sharp rise in the uptake of the yeast coated with OVA protein antigen, enhanced its processing to generate the MHC-II-restricted antigenic epitope and presented it effectively to generate a CD4+ T-cell response.

### Example 5

### Exogenous Administration of HSPs Accelerates Closure of Full Thickness Wounds

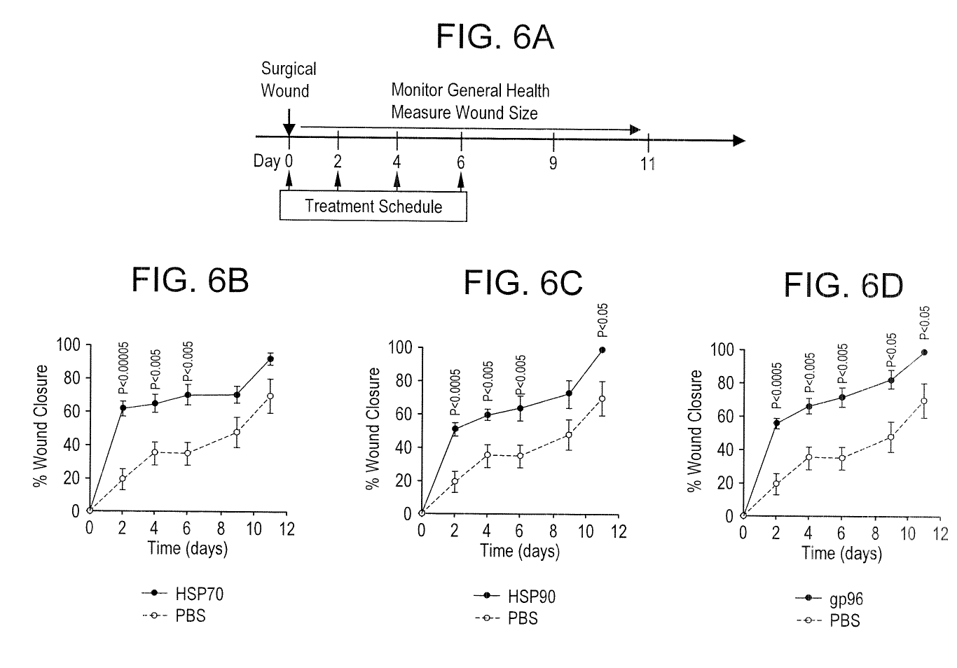

Full thickness surgical wounds were created on the dorsum of BALB/cJ mice as described in Methods. Wounds were made on day 0. Subsequently, the wounds were treated with exogenous injections of HSP70, HSP90, gp96 (30 μg/dose) or buffer administered subcutaneously (SC) on days 0, 2, 4 and 6 following surgery (FIG. 6A). The wounds were measured on days 0, 2, 4, 6, 9, 11 unless otherwise noted. Wounds were measured serially, percent wound closure was calculated and plotted against time in order to quantitate the rate of healing by secondary intention. Wounds in mice treated with HSPs were significantly smaller than in those treated with buffer (FIG. 6 B-D). Data is plotted as average percent wound closure ±SEM on days indicated. Two mice from each treatment group (PBS (n=12), HSP70 (n=11), HSP90 (n=12), and gp96 (n=11)) were sacrificed for histology on days 2, 4, and 6 post-surgery (data not shown). The P values obtain using the Student's t-test. The wounds were completely healed by Day 11 with HSP treatment, whereas controls showed only about 70% healing at the same time point. HSP-treatment increased the rate of healing by secondary intention and reduced the overall time to completion of wound closure. As seen in FIG. 1B-D, HSP-treatment has a rapid onset of wound closure, evident within 48 hours of treatment. Further, HSP-mediated effects on wound closure are sustained throughout the duration of the healing process. Further, the differences in wound size on days 2, 4, 6, 9 and 11 were relatively consistent throughout the healing process (FIG. 6 B-D).

### Example 6

### The Effect of HSPs on Wounds is Protein-Specific and Dose Titratable

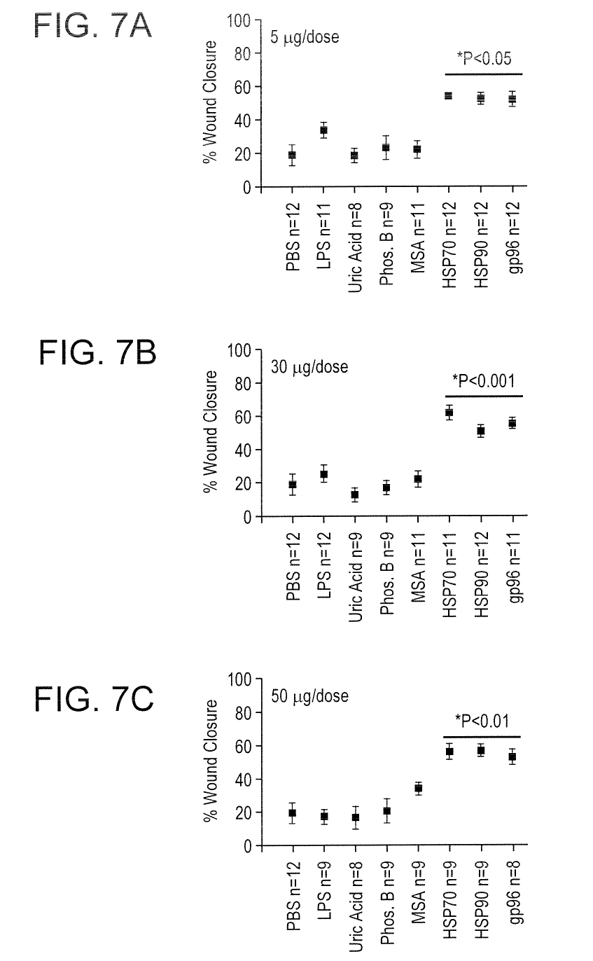

Mice were wounded and treated as described before with either one of the following: non-HSP controls including PBS, LPS, MSA, Uric Acid, or Phosphorylase B or with HSP70, HSP90, gp96 at doses of 5, 30, and 50 μg/dose. At the 5 μg/dose and 50 μg/dose significant wound closure was observed when comparing HSP treatment to controls (FIG. 7 A-C). Results are of average percent wound closure ±SEM on day 2 post surgery. The *P values were obtained using the Student's t-test by comparing HSP treatment to all of the non-HSP control treatments. The least significant P value was reported. The non-HSP controls were not significantly different to the PBS control. Mice receiving doses lower than 5 μg showed no significant wound closure when compared to non-HSP controls (data not shown). Mice receiving doses lower than 5 μg showed no significant wound closure when compared to non-HSP controls (data not shown). Doses higher than 50 μg/dose were not tested. However, at 30 μg/dose the most significant difference in wound closure was observed. On day 2, HSP-treated groups demonstrated a 50-62% wound healing, whereas PBS-treated groups showed 19% healing. Controls including LPS, MSA, uric acid and phosphorylase B showed a 25, 22, 13 and 19% healing, respectively. Besides using the same doses as HSPs themselves, LPS is used in significantly smaller quantities as well. LPS was not beneficial to the wounds at any of the doses tested. At no dose did any of the other non-HSP controls have any significant effect on wound closure.

### Example 7

### The Pro-Inflammatory Cytokine Profile of HSP-Treated Wounds are Unchanged

Pro-inflammatory cytokines and chemokines have been shown to play a major role in wound healing. Since HSPs have been shown to increase production of TNF-α, interleukin (IL)-6, and MCP-1 by macrophages, the ability of exogenously administered HSPs to modulate these cytokines in actual wound tissue was tested (as described in Materials and Methods).

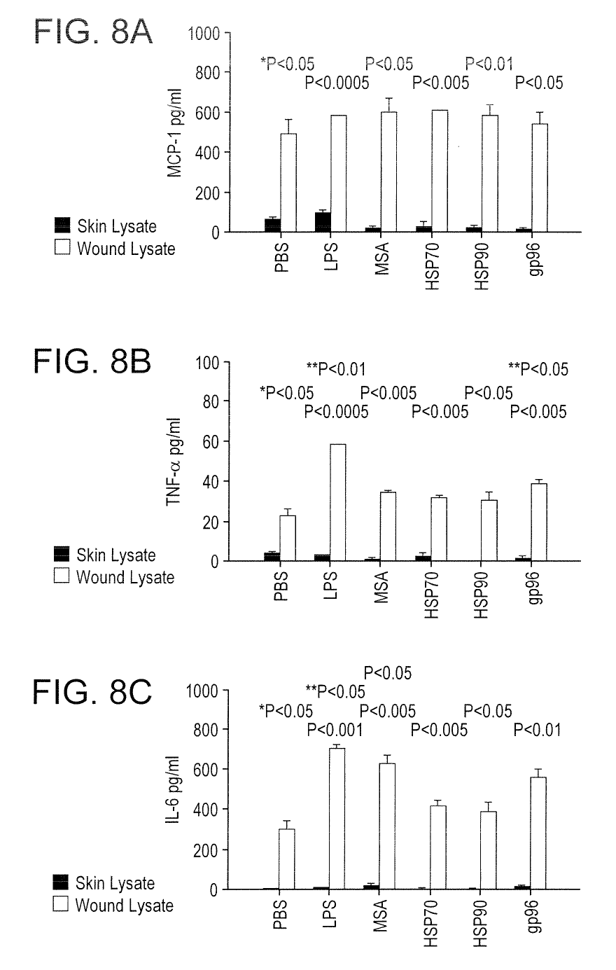

Wounds had significantly higher amounts of IL-6, TNF-α, and MCP-1 when compared to unwounded skin in all treatment groups (FIG. 8A-C). The wounds were lysed and MCP-1, TNFα and IL-6 levels were quantified (pg/ml of 250 μg of protein). Each sample was tested in duplicate. *P values are the significant difference of cytokines found in wound lysate compared to skin lysate in a treatment group. **P values are the significant differences of cytokines found in PBS treated mice wound lysate compared to the treatment group wound lysate.

In wounded animals that were administered SC injections of either one of the HSPs (HSP70, 90 or gp96 30 μg dose) the amount of MCP-1 and IL-6 was similar to that of wounds treated with LPS, MSA, or Buffer, indicating that there was no appreciable increase over basal levels in HSP treated mice. In wounds treated with gp96 the TNF-α content was significantly increased when compared to the buffer group, however was not significantly elevated when compared to LPS or MSA indicating a lack of HSP-specific increase in TNF-α. There was no relevant increase in the levels of the cytokines tested.

### Example 8

### HSP70 Exerts Part of its Effect on Wound Healing Through Macrophages

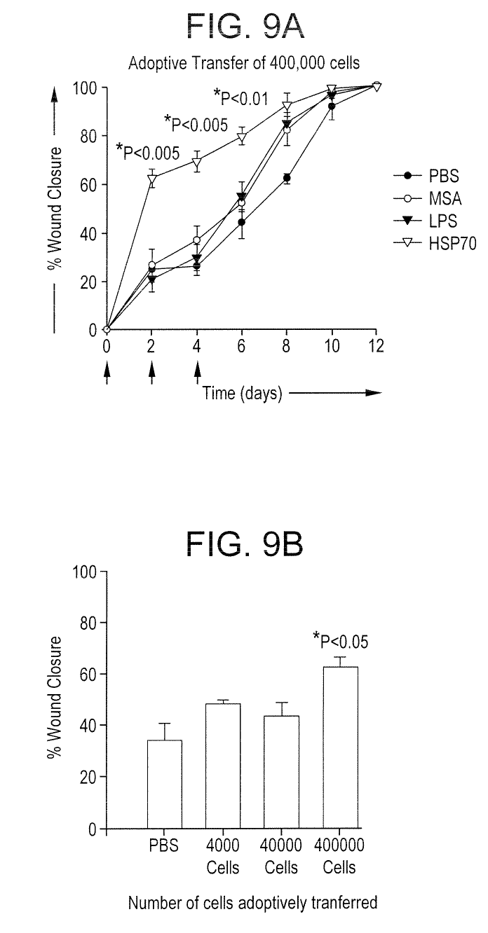

Macrophages were treated in vitro with either HSPs or non-HSP controls and transferred into naïve wounded mice (as described in Materials and Methods) to test if HSPs could in part mediate some of their activity through macrophages. Briefly RAW264.7 cells were treated in vitro with either of HSP70, MSA or LPS, (each at 30 μg/dose) or buffer 1 hour, washed 3 times and administered into naïve mice that had full thickness wounds (as indicated in FIG. 9A) on days 0, 2, and 4 postoperatively. Data is plotted as average percent wound closure ±SEM on day 2. *P values were obtained using the Student's t-test by comparing HSP treatment to all of the non-HSP control treatments. The least significant P value was reported. The non-HSP controls were not significantly different to the PBS control. In order to exclude the possibility the enhanced wound healing was not simply due to a macrophage surplus from the infusion of more macrophages, mice were injected with the same number of macrophages treated with buffer.

Adoptive transfer of 400,000 of macrophages pre-treated with HSP70 (30 μg/dose) significantly accelerated early wound closure whereas macrophages pre-treated with any of the controls did not. Mice given macrophages treated with LPS, MSA, or PBS appeared to have enhanced wound closure (after day 6) when compared to buffer alone (FIG. 9A), however this enhancement was not statistically significant. This indicates that the effect of HSP70 on early wound healing is at least partial mediated by its interaction with macrophages. HSP-mediated effects on wound closure are adoptively transferable via macrophages pre-treated with HSP70 (FIG. 9A). On day 2 post surgery, mice treated with 400,000 cell pretreated with HSP70 were 62% healed whereas wounds in mice adoptively transferred with either PBS, LPS or MSA pre-treated macrophages showed only 25, 21 and 26% healing, respectively. The adoptive transfer of macrophages treated with non-HSP controls is ineffective early demonstrating that the observed effects on early wound closure are HSP-specific. It is inconclusive if these HSP70 treated macrophages influence wound healing at the site of the wound or through a systemic mechanism. Titratability of adoptive transfer was examined by transferring 4,000, 40,000 or 400,000 macrophages pre-treated as described in Materials and Methods. Increasing the number of HSP pretreated cells increases the percentage of wound closure (FIG. 9B).

### Example 9

### HSP70 Partially Accelerates Wound Closure by Upregulating Macrophage-Mediated Phagocytosis

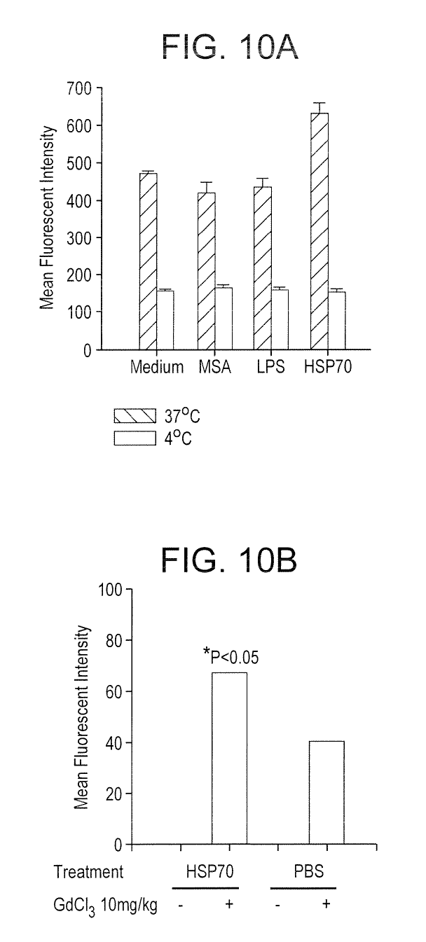

The effect of HSP70 on wound closure is mediated partially via macrophages without an obvious increase in numbers as seen histologically (data not shown). Hence it was deduced that HSP70 mediates its effects by upregulation of macrophage function. Macrophage-mediated phagocytosis has been known to play a fundamental role in wound healing by clearing debris and neutrophils. Therefore it was tested whether HSP70 mediates some of its effects on wound healing through increased macrophage phagocytosis. HSP70 was tested for stimulation of macrophages to increase phagocytic function using wound debris. RAW264.7 macrophages were treated with HSP70, PBS, MSA, or LPS and fed wound debris as stated in Methods. The phagocytosis assay was performed as described previously. Data is plotted as average mean fluorescence intensity from triplicate samples. *P values are the significant difference of mean fluorescence intensity at 37° C. compared to 4° C. in a treatment group. **P values are the significant differences of mean fluorescence intensity of HSP treated macrophages compared to medium treated macrophages. The non-HSP controls were not significantly different to the medium control. HSP 70 treatment stimulated macrophages to phagocytose 25% more wound debris when compared to controls. (FIG. 10 A) This result indicates that HSP70 can directly up-regulate the phagocytic function of macrophages in vitro.

Mice were treated with gadolinium chloride (GdCl₃ (10 mg/kg body weight in 200 μl of distilled water) on Day minus-1 and Day 0. (Intravenous injection of GdCl₃ inhibits phagocytosis of macrophages without affecting their viability. It does not have toxic effects on other cell types). Subsequently the mice were wounded (Day 0) and treated with either PBS or HSP70 (30 μg/dose) given SC on day 0. The percent wound closure in the GdCl₃ treated group was compared with the HSP70 treated group and the percent inhibition of wound closure was calculated (FIG. 10B). Data is shown as the percent inhibition of wound closure by gadolinium chloride. Treatment groups shown are (i) HSP70 alone; (ii) HSP70+GdCl₃; (iii) PBS alone; and (iv) PBS+GdCl₃. *P values were obtained using the Student's t-test by comparing the percent inhibition in wound closure of gadolinium chloride plus treatment to percent wound closure of the treatment alone.

The same was done for the PBS treated group. GdCl₃ caused up to 67% inhibition of the HSP70-mediated effects on wound healing. Since GdCl₃ impairs macrophage function these results show that macrophage functions such as phagocytosis are important for normal wound closure. Most of the HSP70-mediated effects on wound closure are abrogated when macrophage function is impaired, this suggests HSP70 may accelerate wound closure by stimulating increased macrophage function such as phagocytosis; however, HSP70 may work through other mechanisms as well.

### Example 10

### HSP70 Induces Phagocytosis Via Involvement with Toll-Like Receptor 7 (TLR7)

RAW264.7 macrophages in culture were treated with either one of the known ligands of TLR7 including ssPoly(U), ssRNA33, ssRNA40 or LRB (1 mg/ml, each) or medium or with HSP70 (100 μg/ml) alone or with each of the TLR7-ligands named above. Trypan blue exclusion was undertaken to confirm that the cells were viable when treated with either of the agents used. After exposure of these cells for 60 minutes at 37° C., the treated cells were subjected to a phagocytosis assay using Alexa Fluor® 488-labeled Saccharomyces cerevisiae.

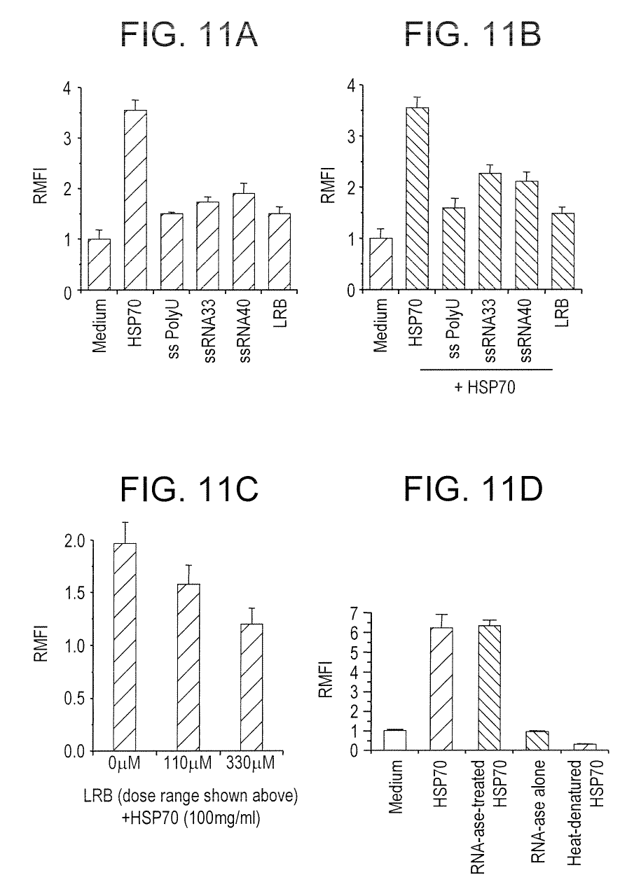

It was observed that all agents enhance phagocytosis of Alexa Fluor® % 488-labeled Saccharomyces cerevisiae by RAW264.7 cells (FIG. 11A). Among the agents tested, HSP70 is the most potent stimulator of phagocytosis. In order to elicit an increase in phagocytosis, the relative dose of HSP70 used (100 μg/ml) was one-tenth of the doses required by the TLR7-ligands. Further, the increase in phagocytosis in response to HSP70-treatment was 4-6 times the basal rate (macrophages treated with medium alone), whereas TLR-ligands induced phagocytosis in the range of 1.5-3 times the basal rate.

Interestingly, when RAW264.7 cells were treated with HSP70 in the presence of known TLR7-ligands (FIG. 11B), a significant decrease (range 45-65%) in the phagocytic activity was observed as compared to the phagocytosis seen by RAW264.7 cells treated with HSP70 alone. Using the (analysis of variance) ANOVA single factor test the p value for HSP70 treatment alone as compared with any of the TLR7-ligands was <0.01. The corresponding decrease in phagocytosis was somewhat equivalent to that seen with the phagocytosis when ligands were used alone, (as shown in FIG. 11A). In order to examine whether the interaction of TLR7 ligands with HSP70 was titratable by dose, the following experiment was performed. RAW264.7 cells were treated with HSP70 (100 μg/ml) in the presence of varying doses of Loxoribine (LRB), a TLR7-ligand (range: 0-330 μM concentration) and a phagocytosis assay was performed. As seen in FIG. 11C the inhibitory effect of LRB on HSP70-mediated phagocytosis is titratable by dose.

Viral RNA, including single-stranded (ssRNA), is known to bind HSP70. SsRNA is also a known ligand for TLR7; hence, the possibility that contaminating ssRNA in the HSP70-preparation is responsible for the activity in FIGS. 11A and B was considered. RAW264.7 were treated with HSP70 (100 μg/ml) alone or with HSP70 pre-treated with RNase (200 μg/ml for 30 minutes at room temperature (RT)). In order to ensure that RNase was effective in the conditions used, a known quantity of ssRNA33 was treated with the RNase (200 μg/ml for 30 minutes at room temperature) and was noted to be completely digested indicating that the conditions and the RNase used was effective. (Data not shown). A phagocytosis assay using Alexa Fluor® 488-labeled Saccharomyces cerevisiae (as described in Materials and Methods) (FIG. 11D) showed that treatment of HSP70 pre-treated with RNAase had no influence on its ability to stimulate phagocytosis by RAW264.7 cells (p>0.05).

It has been shown herein that uncomplexed HSPs can be administered therapeutically to a vertebrate organism with an active infection or prophylactically to a vertebrate organism at risk of future infection with a harmful agent. Previously, HSP-peptide complexes have been employed in methods such as pathogen elimination and therapy for infections. Unexpectedly, uncomplexed HSPs activate phagocytosis by macrophages, and are thus particularly useful for the rapid clearance of harmful particulate agents including pathogenic organisms and particulate pollutants. A major advantage of this discovery is that the therapy is non-specific, that is, the pathogen need not be identified prior to treatment with uncomplexed HSP. Thus, treatment with uncomplexed HSPs can be used as a first line treatment that can be administered even prior to identification of the particular pathogen or agent. Use of uncomplexed HSPs to stimulate an immune response has particular utility in the treatment of unidentified infections, emergent infections, antibiotic-resistant infections, infections caused by pathogens or other harmful agents that are difficult to detect, invasive fungal infections, and other infections.

Another advantage of therapy with HSPs is that some patients are allergic to conventional anti-infectives, the very compounds necessary to treat their infection. For these patients, only few drugs might be available to treat the infection. If the patient is infected with a strain of bacteria that does not respond well to substitute therapies, the patient's life can be in danger.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

All cited patents, patent applications, and other references are incorporated herein by reference in their entirety.

1. A method of promoting pathogen elimination from a vertebrate organism, comprising:
administering a phagocytosis-stimulating amount of an uncomplexed heat shock protein to the vertebrate organism in need of such administration,
wherein administering is therapeutic or prophylactic.

2. The method of claim 1, wherein the vertebrate organism has an infection caused by the pathogen, has been exposed to the pathogen, or is suspected of exposure to the pathogen.

3. The method of claim 1, wherein the pathogen comprises an NIAID category A, B, and C priority pathogen; an unidentified pathogen; an emergent pathogen; an antibiotic-resistant pathogen; a pathogen that is difficult to detect; or a combination comprising one or more of the foregoing pathogens.

4. The method of claim 1, wherein the vertebrate organism has an infection caused by the pathogen.

5. The method of claim 4, wherein the infection is a systemic infection, a deep tissue infection, or an invasive fungal infection.

6. The method of claim 1, wherein the uncomplexed heat shock protein comprises HSP70, HSP90, gp96, or a combination of one or more of the foregoing heat shock proteins.

7. The method of claim 1, wherein administering is performed as a first line treatment after infection.

8. The method of claim 7, wherein first line treatment is performed prior to identification of the pathogen.

9. The method of claim 1, wherein the vertebrate organism has an infected wound site.

10. The method of claim 9, wherein the infected wound site comprises a tissue damaged by traumatic injury, a tissue damaged by severe diabetic infection, or a tissue damaged by a candidal infection.

11. A method of reducing infection, necrotic tissue, or both at a wound site in a vertebrate organism, comprising
administering a phagocytosis-stimulating amount of an uncomplexed heat shock protein to the vertebrate organism in need of such administration,
wherein the vertebrate organism has an infected wound, a chronic wound comprising necrotic tissue, or a combination thereof.

12. The method of claim 11, wherein the uncomplexed heat shock protein comprises HSP70, HSP90, gp96, or a combination of one or more of the foregoing heat shock proteins.

13. The method of claim 11, wherein the wound site is an infected wound site and administering is performed prior to identification of the pathogen.

14. A method of promoting particulate agent elimination from a vertebrate organism, comprising:
administering a phagocytosis-stimulating amount of an uncomplexed heat shock protein to the vertebrate organism after exposure or suspected exposure to the particulate agent.

15. The method of claim 14, wherein the particulate agent is a particulate pollutant.

16. The method of claim 15, wherein the particulate pollutant comprises asbestos, cadmium, mercury, silica, coal dust, carbon, tin oxide, beryllium, stone dust, graphite, or a combination of one or more of the foregoing pollutants.

17. The method of claim 14, wherein the particulate pollutant comprises a biological agent.

18. The method of claim 17, wherein the biological agent comprises a bacterium, a virus, a toxin, or a combination thereof.

19. The method of claim 14, wherein administering is performed prior to identification of the particulate agent.

20. The method of claim 14, wherein the uncomplexed heat shock protein comprises HSP70, HSP90, gp96, or a combination of one or more of the foregoing heat shock proteins.


SEQUENCE LISTING (9 sequences)

SEQ ID NO: 1 - DNA, 2691 bp, Homo sapiens
cgccatggagaccaacacccttcccaccgccactcccccttcctctcagggtccctgtcccctccagtgaatcccagaagactctggagagttctgagcagggggcggcactctggcctctgattggtccaaggaaggctggggggcaggacgggaggcgaaacccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagccgagccgacagagagcagggaaccgcatggccaaagccgcggcagtcggcatcgacctgggcaccacctactcctgcgtgggggtgttccaacacggcaaggtggagatcatcgccaacgaccagggcaaccgcaccacccccagctacgtggccttcacggacaccgagcggctcatcggggatgcggccaagaaccaggtggcgctgaacccgcagaacaccgtgtttgacgcgaagcgcctgatcggccgcaagttcggcgacccggtggtgcagtcggacatgaagcactggcctttccaggtgatcaacgacggagacaagcccaaggtgcaggtgagctacaagggggagaccaaggcattctaccccgaggagatctcgtccatggtgctgaccaagatgaaggagatcgccgaggcgtacctgggctacccggtgaccaacgcggtgatcaccgtgccggcctacttcaacgactcgcagcgccaggccaccaaggatgcgggtgtgatcgcggggctcaacgtgctgcggatcatcaacgagcccacggccgccgccatcgcctacggcctggacagaacgggcaagggggagcgcaacgtcctgatctttgacctgggcgggggcaccttcgacgtgtccatcctgacgatcgacgacggcatcttcgaggtgaaggccacggccggggacacccacctgggtggggaggactttgacaacaggctggtgaaccacttcgtggaggagttcaagagaaaacacaagaaggacatcagccagaacaagcgagccgtgaggcggctgcgcaccgcctgcgagagggccaagaggaccctgtcgtccagcacccaggccagcctggagatcgactccctgtttgagggcatcgacttctacacgtccatcaccagggcgaggttcgaggagctgtgctccgacctgttccgaagcaccctggagcccgtggagaaggctctgcgcgacgccaagctggacaaggcccagattcacgacctggtcctggtcgggggctccacccgcatccccaaggtgcagaagctgctgcaggacttcttcaacgggcgcgacctgaacaagagcatcaaccccgacgaggctgtgggctacggggcggcggtgcaggcggccatcctgatgggggacaagtccgagaacgtgcaggacctgctgctgctggacgtggctcccctgtcgctggggctggagacggccggaggcgtgatgactgccctgatcaagcgcaactccaccatccccaccaagcagacgcagatcttcaccacctactccgacaaccaacccggggtgctgatccaggtgtacgagggcgagagggccatgacgaaagacaacaatctgttggggcgcttcgagctgagcggcatccctccggccccaggcgtgccccagatcgaggtgaccttcgacatcgatgccaacggcatcctgaacgtcacggccacggacaagagcaccggcaaggccaacaagatcaccatcaccaacgacaagggccgcctgagcaaggaggagatcgagcgcatggtgcaggaggcggagaagtacaaagcggaggacgaggtgcagcgcgagagggtgtcagccaagaacgccctggagtcctacgccttcaacatgaagagcgccgtggaggatgaggggctcaagggcaagatcagcgaggccgacaagaagaaggtgctggacaagtgtcaagaggtcatctcgtggctggacgccaacaccttggccgagaaggacgagtttgagcacaagaggaaggagctggagcaggtgtgtaaccccatcatcagcggactgtaccagggtgccggtggtcccgggcctgggggcttcggggctcagggtcccaagggagggtctgggtcaggccccaccattgaggaggtagattaggggcctttccaagattgctgtttttgttttggagcttcaagactttgcatttcctagtatttctgtttgtcagttctcaatttcctgtgtttgcaatgttgaaattttttggtgaagtactgaacttgcctttttttccggtttctacatgcagagatgaatttatactgccatcttacgactatttcttctttttaatacacttaactcaggccattttttaagttggttacttcaaagtaaataaactttaaaattcaagtgatgcccttttattcct

SEQ ID NO: 2 - DNA, 2912 bp, Homo sapiens
cagttgcttcagcgtcccggtgtggctgtgccgttggtcctgtgcggtcacttagccaagatgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaactttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgatctgtttgaaaacagaaagaaaaagaacaatatcaaattgtatgtacgcagagttttcatcatggataactgtgaggagctaatccctgaatatctgaacttcattagaggggtggtagactcggaggatctccctctaaacatatcccgtgagatgttgcaacaaagcaaaattttgaaagttatcaggaagaatttggtcaaaaaatgcttagaactctttactgaactggcggaagataaagagaactacaagaaattctatgagcagttctctaaaaacataaagcttggaatacacgaagactctcaaaatcggaagaagctttcagagctgttaaggtactacacatctgcctctggtgatgagatggtttctctcaaggactactgcaccagaatgaaggagaaccagaaacatatctattatatcacaggtgagaccaaggaccaggtagctaactcagcctttgtggaacgtcttcggaaacatggcttagaagtgatctatatgattgagcccattgatgagtactgtgtccaacagctgaaggaatttgaggggaagactttagtgtcagtcaccaaagaaggcctggaacttccagaggatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaagagacaactcaacaatgggttacatggcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtattgatgaagatgaccctactgctgatgataccagtgctgctgtaactgaagaaatgccaccccttgaaggagatgacgacacatcacgcatggaagaagtagactaatctctggctgagggatgacttacctgttcagtactctacaattcctctgataatatattttcaaggatgtttttctttatttttgttaatattaaaaagtctgtatggcatgacaactactttaaggggaagataagatttctgtctactaagtgatgctgtgataccttaggcactaaagcagagctagtaatgctttttgagtttcatgttggttctttcacagatggggtaacgtgcactgtaagacgtatgtaacatgatgttaactttgtgtggtctaaagtgtttagctgtcaagccggatgcctaagtagaccaaatcttgttattgaagtgttctgagctgtatcttgatgtttagaaaagtattcgttacatcttgtaggatctactttttgaacttttcattccctgtagttgacaattctgcatgtactagtcctctagaaataggttaaactgaagcaacttgatggaaggatctctccacagggcttgttttccaaagaaaagtattgtttggaggagcaaagttaaaagcctacctaagcatatcgtaaagctgttcaaatactcgagcccagtcttgtggatggaaatgtagtgctcgagtcacattctgcttaaagttgtaacaaatacagatgagt

SEQ ID NO: 3 - DNA, 1184 bp, Homo sapiens
acaatggagatcctggaataggcacatatctctacgtaatcagaacgagatgtaacatctaagacgcagggcagggctctggcaaagacacgctgggggaacgtgaagaaagggccaaaactacgcagctggtcctcagagaccaaagactacgacccccagcattcaatgcagtgaacccctaggacgttgcatgccgggagctgtagtttcatcaccaccgccacccccccgcccccccgccatctgaaagcgttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgccatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgtgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgggaagtgggggtgaaaagcggcccgacctgcttgcggtgtagtgggcggaccgcgcggctggaggtgtgaggatccgaacccaggggtggggggtggaggcggctcctgcgatcgaaggggacttgagactcaccggccgcacgccatgagggccctgtgggtgctgggcctctgctgcgtcctgctgaccttcggtgagtgattctggaggagcagacgtcccccctccacacacgcggccgcttctcgaaggtcctgggggcgttgaacgtgggaggggggatcccggggcctgcggtgggccaagggacgtcaccattcctcgaaagagggcaagggaattacgtttattctcttcttcctctggaaataaaaaaagagagcaacatcatctaactgccctacagatctaatagtatctc

SEQ ID NO: 4 - DNA, 2780 bp, Homo sapiens
gtgggcggaccgcgcggctggaggtgtgaggatccgaacccaggggtggggggtggaggcggctcctgcgatcgaaggggacttgagactcaccggccgcacgccatgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaaaagatgaattgtaaattatactctcaccatttggatcctgtgtggagagggaatgtgaaatttacatcatttctttttgggagagacttgttttggatgccccctaatccccttctcccctgcactgtaaaatgtgggattatgggtcacaggaaaaagtgggttttttagttgaattttttttaacattcctcatgaatgtaaatttgtactatttaactgactattcttgatgtaaaatcttgtcatgtgtataaaaataaaaaagatcccaaat

SEQ ID NO: 5 - DNA, 2233 bp, Homo sapiens
gggacggcgacagcgggtcggcgggccgcaggagggggtcatgggtaaagactactaccagacgttgggcctggcccgcggcgcgtcggacgaggagatcaagcgggcctaccgccgccaggcgctgcgctaccacccggacaagaacaaggagcccggcgccgaggagaagttcaaggagatcgctgaggcctacgacgtgctcagcgacccgcgcaagcgcgagatcttcgaccgctacggggaggaaggcctaaaggggagtggccccagtggcggtagcggcggtggtgccaatggtacctctttcagctacacattccatggagaccctcatgccatgtttgctgagttcttcggtggcagaaatccctttgacaccttttttgggcagcggaacggggaggaaggcatggacattgatgacccattctctggcttccctatgggcatgggtggcttcaccaacgtgaactttggccgctcccgctctgcccaagagcccgcccgaaagaagcaagatcccccagtcacccacgaccttcgagtctcccttgaagagatctacagcggctgtaccaagaagatgaaaatctcccacaagcggctaaaccccgacggaaagagcattcgaaacgaagacaaaatattgaccatcgaagtgaagaaggggtggaaagaaggaaccaaaatcactttccccaaggaaggagaccagacctccaacaacattccagctgatatcgtctttgttttaaaggacaagccccacaatatctttaagagagatggctctgatgtcatttatcctgccaggatcagcctccgggaggctctgtgtggctgcacagtgaacgtccccactctggacggcaggacgatacccgtcgtattcaaagatgttatcaggcctggcatgcggcgaaaagttcctggagaaggcctccccctccccaaaacacccgagaaacgtggggacctcattattgagtttgaagtgatcttccccgaaaggattccccagacatcaagaaccgtacttgagcaggttcttccaatatagctatctgagctccccaaggactgaccagggacctttccagagctcaaggatttctggacctttctaccagttgtggaccatgagagggtgggagggcccagggagggctttcgtactgctgaatgttttccagagcatatattacaatctttcaaagtcgcacactagacttcagtggtttttcgagctatagggcatcaggtggtgggaacagcaggaaaaggcattccagtctgccccactgggtctggcagccctcccgggatgggcccacatccacctccagtccctggccaggggtgagaggcagaccagcagatggacttgatccctctgtgtctttgggcttctggctggtagataatgtcaacctgcagtcttgattcccagaccctgtacactcctccttttctgttgtgtgatcagtttgtgctttattctgtatttgtctcccatgtcttgctcttctcctggagaattctgtcttctctttggccatctcaaattgagaacctaaactattcctgcagaactgcctggttggcgtccacaagcaatacctctcgttccagcaggaccaagggagccagcctccagtgagtgactccagcaagtgcagccacctctcccttgatggtctgggagcctggcctcagcaaggggccttcctgacctctggctccagtgaagctgaatgtcctcactttgtgggtcacactctttacatttctgtaaggcaatcttggcacacgtggggcttaccagtggcccaggtaattttttgtttcatggactatggactctttcaaagggatctgatccttttgaattttgcacagccctagatacaatcccttttgataaaagggtctttgcttctgattacaggagcactgtggaacgtctgtaaatatgtttttataattccatgtatagttggtgtacactcaaaacctgtccccggcagccagtgctctctgtatagggccataatggaattctgaagaaatcttggggagggaaggggagttggaacaaatgtctgttccctggaggccagtccagtgctcagacctttagactcattgtaagttgccactgccaacatgagaccaaagtgtgtgactagtcaatgaagtgcgacagcattaaagactgatgctaaacctc

SEQ ID NO: 6 - DNA, 5470 bp, Homo sapiens
cccggggtcactcctgctggacctactccgaccccctaggccgggagtgaaggcgggacttgtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcgaaattgcgctgtgctcctgtgctacggcctgtggctggactgcctgctgctgcccaactggctggcaagatgaagctctccctggtggccgcgatgctgctgctgctcagcgcggcgcgggccgaggaggaggacaagaaggaggacgtgggcacggtggtcggcatcgacttggggaccacctactcctggtaagtggggttgcggatgagggggacggggcgtggcgctggctggcgtgagaagtgcggtgctgatgtccctctgtcgggtttttgcagcgtcggcgtgttcaagaacggccgcgtggagatcatcgccaacgatcagggcaaccgcatcacgccgtcctatgtcgccttcactcctgaaggggaacgtctgattggcgatgccgccaagaaccagctcacctccaaccccgagaacacggtctttgacgccaagcggctcatcggccgcacgtggaatgacccgtctgtgcagcaggacatcaagttcttgccgttcaaggttcgaccggttttcctcatccagttagagaacgggtgggtggtgggagtatttagagttataagtctctggaaaagtgttgagacaacagttgaaggttatagacatgatgtatgtaataactttaatactattagtatgttacaaaacttaagacagttgctgtcgtactgtctacgatagtttaggaataaaagaccgattaaaactgaactttgtaagacacctatactccctgaagtatttctagtcaatttgcagccccaagggaccaaaataaaccaaattgtggggatggtagtgggtcttttaaactttgagatgtcattgtatctgtgtctgaaaacaataattctttaaaataggtggttgaaaagaaaactaaaccatacattcaagttgatattggaggtgggcaaacaaagacatttgctcctgaagaaatttctgccatggttctcactaaaatgaaagaaaccgctgaggcttatttgggaaagaaggtaaatatttctagaacaatgttaagtattttttgatcattagtattctcggttggctgttatgtatagaagccttcgtgaagggtttcaaaaattttaatcagaatggtattcatgcttgtcacggtttaattattgagtccctttactataagccaaacaaaaatagacttttcatgtattatttaatgcttacaattccaggaacaataaaattttatatgttgtattcatcaataattggcttaaaaactaaagtgatggtttgactgtaattttttttttttgagatggagtcttgctctgttgcccaggctggactgcagtggcacgatctcagctcactgcaacctctgcctcccgggttaagcagctctcctgcctcagcctccaagtaatggaacgacaggcacaccaccacagctggctaatttttttttttttttttaattttcagtagagacagggtttctccacattgccaggctggtcttgaaatcctgccctcaggttgatcctcctgcctagcctcccaaagtgctggattataggcagaagccaccgcctggccagactgtaatttaaataagggttaaactatgtgacaatacacttaattatctttatccttttaggttacccatgcagttgttactgtaccagcctattttaatgatgcccaacgccaagcaaccaaagacgctggaactattgctggcctaaatgttatgaggatcatcaacgagccgtaagtatgaaattcagggatacggcatatttgccaaatagtggaaatgtgaagtactgacaaaacttttccctttttcaatctaatagtacggcagctgctattgcttatggcctggataagagggagggggagaagaacatcctggtgtttgacctgggtggcggaaccttcgatgtgtctcttctcaccattgacaatggtgtcttcgaagttgtggccactaatggagatactcatctgggtggagaagactttgaccagcgtgtcatggaacacttcatcaaactgtacaaaaagaagacgggcaaagatgtcaggaaggacaatagagctgtgcagaaactccggcgcgaggtagaaaaggccaaggccctgtcttctcagcatcaagcaagaattgaaattgagtccttctatgaaggagaagacttttctgagaccctgactcgggccaaatttgaagagctcaacatggtatgttccttgttttctgctttgctaatgagatctccttagactctgaattcaggacattgcatctagatacttagataacagacatcacagtaaccatgtcttttttctaggatctgttccggtctactatgaagcccgtccagaaagtgttggaagattctgatttgaagaagtctgatattgatgaaattgttcttgttggtggctcgactcgaattccaaagattcagcaactggttaaagagttcttcaatggcaaggaaccatcccgtggcataaacccagatgaagctgtagcgtatggtgctgctgtccaggctggtgtgctctctggtgatcaagatacaggtaggtcatcatcgcagcatctttcttagtgattcagtagcttgatggaagagctcggtacccctattgctttagaaaataccagaatatgagcaacaaggtcacacagctagtaaagggtataagtgaagacaagactggggtagtctccaagatcattagcaactgtttaattcactgcctttaaaatgtgtgtgttagaacctaaccaaatgttagagagataaactttacatagctcatagggagaacttgaattaaaagttaaataacttatccttacaggtgacctggtactgcttcatgtatgtccccttacacttggtattgaaactgtaggaggtgtcatgaccaaactgattccaagtaatacagtggtgcctaccaagaactctcagatcttttctacagcttctgataatcaaccaactgttacaatcaaggtctatgaaggtaattaccttaagtttggttaatatcatggctttttttttgagatgaagtcttgctctgttgcccaggctggactgcagtggcacgatctcggctcactgcaaattctgtctcccgggttcaagtgattctcctgcctcagcctccagagtagctggattacagcctgaccaccacacctggctaatttctgtatttttagtagaggatgggctttcaccatgtttcccaggctggtctccaactcctgacctcaggtcatctgcctgcctccaccgtcccgaaagtactgggattatagcgtgagccaccacgccagatctatctatcatggcatattttaaaagaacatgacttaatatgtcctattgaaatggctagggaactaagtaactgctgttttcagatggaggtcttaatttgaataatgttgatattagatatttagcattcttttttttttttttttaatggagtcttgctctgtcgcctaggctggggtgcagtggcatgacttgcaacctctgcctcccgaatagctgggattacaggtgcccaccatcacgcccggctaagttttgtatttttagtagaggcgagtttcgccatgttggccaggctggtcttgaacccctaacctcagtgatcccacggtcaccgacctggcctcccaaaagtactgtacccagccaatgattagcattctcactaataatagcatctgagctggctcctagagtacaagaaaaaggagttcacagtactttaaaatagataaaattcagttgagttagtaacctaactcattgttagtactagttgctgctccttgtagaccaatatgaaattacttttagctcgataaaaccaaaagtgtcactttatgcttcagactgaaatgcggggatctagatgtgctaatgcttgtcagtaacaactaacaagtttttctgtatgtaacttctaggtgaaagacccctgacaaaagacaatcatcttctgggtacatttgatctgactggaattcctcctgctcctcgtggggtcccacagattgaagtcacctttgagatagatgtgaatggtattcttcgagtgacagctgaagacaagggtacagggaacaaaaataagatcacaatcaccaatgaccagaatcgcctgacacctgaagaaatcgaaaggatggttaatgatgctgagaagtttgctgaggaagacaaaaagctgaaggagcgcattgatactagaaatgagttggaaagctatgcctattctctaaagaatcagattggagataaagaaaagctgggaggtaaactttcctctgaagataaggagaccatggaaaaagctgtagaagaaaagattgaatggctggaaagccaccaagatgctgacattgaagacttcaaagctaagaagaaggaactggaagaaattgttcaaccaattatcagcaaactctatggaagtgcaggccctcccccaactggtgaagaggatacagcagaaaaagatgagttgtagacactgatctgctagtgctgtaatattgtaaatactggactcaggaacttttgttaggaaaaaattgaaagaacttaagtctcgaatgtaattggaatcttcacctcagagtggagttgaaactgctatagcctaagcggctgtttactgcttttcattagcagttgctcacatgtctttgggtgggggggagaagaagaattggccatcttaaaaagcgggtaaaaaacctgggttagggtgtgtgttcaccttcaaaatgttctatttaacaactgggtcatgtgcatctggtgtaggaggttttttctaccataagtgacaccaataaatgtttgttatttacactggtctaatgtttgtgagaagctt

SEQ ID NO: 7 - DNA, 313 bp, Mus musculus
ggcggctcctgagaccgaaaaggacttgcgactcgccggccacgcacatgagggtcctgtgggtgttgggcctctgctgtgtcctgctgaccttcgggttcgtcagagctgatgatgaagtcgacgtggatggcacagtggaagaggacctgggtaaaagccgagaaggctcaaggacagatgatgaagttgtgcagagagaggaagaagctattcagttggatgggttaaacgcatcacagataagagaacttagagaaaaatctgaaaagttcgccttccaagctgaagtgaacaggatgatgaaacttat

SEQ ID NO: 8 - DNA, 624 bp, Mus musculus
cggttaccggcggaaacggtctcggggtgagaggtcacccgaagacaggcagctgctgaaccaataggaccagcgctcagggcggatcgtgcctctcattggtggccgttaagaatgaccagtagccaatgagtcagcccggggggcgtagcaatgacgtgagttgcggaggaggccgcttcgaatcggcagcagccagcttggtggcatggaccaatcagcggcctccaactagtagcgacttcaccaatcggaggcctccacgacggggctgtggggagggtatataaggcgagtcggcgacggcgcgctcgatactggccgagacaacactgacctggacacttgggcttctgcgtgtgtgtgaggtaagcgccgcggcctgctgctaggcctgctccgagtctgctcgtgtctcctcctgaccccgaggcccctgtcgccctcagaccagaaccgtcgtcgcgtttcgggccacagcctgttgctggactcctaagactcctgcctgactgctgagcgactggtcctcagcgccggcatgatgaagttcactgtggtggcggcggcgttgctgctgctgggcgcggtgcgggccgaggaggaggacaagaaggaggatgt

SEQ ID NO: 9 - DNA, 3518 bp, Mus musculus
gatctcttctatttccctattcaaacctaaaatgaagagggagggggagacatggacaagcaagcattccacaggcgcccctgcccaacgctgtcactcaaaccaggacccaatcacagactttttagccaagccttatcccgcctctcttgagaaactttctgcgtccgccatcctgtaggaaggatttgtacactttaaactccctccctggtctgagtcccacactctcaccacccagcaccttcaggagctgacccttaacagcttcacccacagggaccccgaagttgcgtcgcctccgcaacagtgtcaatagcagcaccagcacttccccacaccctccccctcaggaatccgtactctctagcgaaccccagaaacctctggagagttctggacaagggcggaacccacaactccgattactcaagggaggcggggaagctccaccagacgcgaaactgctggaagattcctggccccaaggcctcctccggctcgctgattggcccagcggagagtgggcggggccggtgaagactccttaaaggcgcagggcggcgagcagggcaccagacgctgacagctactcagaatcaaatctggttccatccagagacaagcgaagacaagagaagcagagcgagcggcgcgttcccgatcctcggccaggaccagccttccccagagcatccacgccgcggagcgcaaccttcccaggagcatccctgccgcggagcgcaactttccccggagcatccacgccgcggagcgcagccttccagaagcagagcgcggcgccatggccaagaacacggcgatcggcatcgacctgggcaccacctactcgtgcgtgggcgtgttccagcacggcaaggtggagatcatcgccaacgaccagggcaaccgcacgacccccagctacgtggccttcaccgacaccgagcgcctcatcggggacgccgccaagaaccaggtggcgctgaacccgcagaacaccgtgttcgacgcgaagcggctgatcggccgcaagttcggcgatgcggtggtgcagtccgacatgaagcactggcccttccaggtggtgaacgacggcgacaagcccaaggtgcaggtgaactacaagggcgagagccggtcgttcttcccggaggagatctcgtccatggtgctgacgaagatgaaggagatcgctgaggcgtacctgggccacccggtgaccaacgcggtgatcacggtgcccgcctacttcaacgactctcagcggcaggccaccaaggacgcgggcgtgatcgccggtctaaacgtgctgcggatcatcaacgagcccacggcggccgccatcgcctacgggctggaccggaccggcaagggcgagcgcaacgtgctcatcttcgacctggggggcggcacgttcgacgtgtccatcctgacgatcgacgacggcatcttcgaggtgaaggccacggcgggcgacacgcacctgggaggggaggacttcgacaaccggctggtgagccacttcgtggaggagttcaagaggaagcacaagaaggacatcagccagaacaagcgcgcggtgcggcggctgcgcacggcgtgtgagagggccaagaggacgctgtcgtccagcacccaggccagcctggagatcgactctctgttcgagggcatcgacttctacacatccatcacgcgggcgcggttcgaagagctgtgctcggacctgttccgcggcacgctggagcccgtggagaaggccctgcgcgacgccaagatggacaaggcgcagatccacgacctggtgctggtgggcggctcgacgcgcatccccaaggtgcagaagctgctgcaggacttcttcaacgggcgcgacctgaacaagagcatcaacccggacgaggcggtggcctacggggcggcggtgcaggcggccatcctgatgggggacaagtcggagaacgtgcaggacctgctgctgctggacgtggcgccgctgtcgctgggcctggagactgcgggcggcgtgatgacggcgctcatcaagcgcaactccaccatccccaccaagcagacgcagaccttcaccacctactcggacaaccagcccggggtgctgatccaggtgtacgagggcgagagggccatgacgcgcgacaacaacctgctggggcgcttcgagctgagcggcatcccgccggcgcccaggggcgtgccgcagatcgaggtgaccttcgacatcgacgccaacggcatcctgaacgtcacggccaccgacaagagcaccggcaaggccaacaagatcaccatcaccaacgacaagggccgcctgagcaaggaggagatcgagcgcatggtgcaggaggccgagcgctacaaggccgaggacgaggtgcagcgcgacagggtggccgccaagaacgcgctcgagtcctatgccttcaacatgaagagcgccgtggaggacgagggtctcaagggcaagctcagcgaggctgacaagaagaaggtgctggacaagtgccaggaggtcatctcctggctggactccaacacgctggccgacaaggaggagttcgtgcacaagcgggaggagctggagcgggtgtgcagccccatcatcagtgggctgtaccagggtgcgggtgctcctggggctgggggcttcggggcccaggcgccgccgaaaggagcctctggctcaggacccaccatcgaggaggtggattagaggcctctgctggctctcccggtgtggtctagaaaacagactctttgcacttgatagctgcttgggcaccgattactgtcaaggttatttaaagtcttcttcatggttcagtttaaagttacagtctttcttaaggtaattgcgttgactgttaaattttgtatgcatatatatatatatatatatatatatatatatattcaaatatattcaaagtaatgttgggagcagcactgtgcactgtaccaggggattatgttttatagctaatgatgtgtaaagtctaaagatttttttgtaatttttatatcagtgttccagtagcctgggaagacatatagtctagctgcccagttccctggagatggtcatctctaagacaaagtgtcttaaacaaacgtcttggcactgtgtactacataactttactcttttgtacttaaaactttatctgcttgtccatgttaaggttttgtggtataaccagtatgttctttgcatttaatctaagtaggttaaagatggtgtatccttcctgcatacatgtctacactgccaccctgtgtacatttttttctttgcatcactacaaactaatgaaaaaaacttttatgacttaaatattcaaaataaaaggttacaagtatattttgtctgtttgtatgttggaagggctaatggattctgggcttctgtggatttcttaagttttttttaagatttattattatatgtgaacacattgtagctatcttcagacacaccagaaaagggcatcagatct